(19) United States
(12) Patent Application Publication
(10) Pub. No.: US 2015/0018398 A1
(43) Pub. Date: Jan. 15, 2015

(54) METHODS OF USING ESTROGEN RECEPTOR-BETA LIGANDS AS RADIATION MITIGATORS

(71) Applicant: The Regents of the University of California, Oakland, CA (US)

(72) Inventors: Richard J. Pietras, Los Angeles, CA (US); Michael E. Jung, Los Angeles, CA (US)

(21) Appl. No.: 14/332,204

(22) Filed: Jul. 15, 2014

Related U.S. Application Data
(60) Provisional application No. 61/846,498, filed on Jul. 15, 2013.

(51) Int. Cl. A61K 31/423; A61K 31/277

(52) U.S. Cl. CPC A61K 31/423; A61K 31/277
USPC 514/375; 514/520

(57) ABSTRACT

Disclosed herein are methods useful for treating radiation damage in a subject.

### CROSS-REFERENCES TO RELATED APPLICATIONS

This application claims the benefit of U.S. Provisional Application No. 61/846,498, filed Jul. 15, 2013, the content of which is incorporated by reference herein and for all purposes.

### STATEMENT AS TO RIGHTS TO INVENTIONS MADE UNDER FEDERALLY SPONSORED RESEARCH AND DEVELOPMENT

This invention was made with government support under grant nos. U19 A1-67769, awarded by the National Institutes of Health. The government has certain rights in the invention.

### BACKGROUND

The threat of nuclear or radiological attacks in the United States has grown recently due to increased activity of terrorist groups and illicit trafficking of radioactive materials. Further, recent natural disasters, such as the devastating earthquakes in Japan which threatened the security of nuclear power facilities, highlight the need to develop and stockpile countermeasures to treat radiation injuries in the general population. At this time, there are few approved medical agents available to manage the variety of short- and long-term injuries that can result from nuclear or radiological attacks. To expand the options available to mitigate and/or treat radiation-induced injury, new translational research is needed.

Injuries to bone marrow, gastrointestinal tract, respiratory and cardiovascular systems are major determinants of lethality after total-body irradiation (TBI). Although some progress has been made in the management of systemic radiation injury, development of additional effective and safe countermeasures against structural injury and dysfunction remain an urgent need, especially in view of increasing risks of nuclear or radiological accidents or attacks. To be useful in an actual mass casualty situation, a medial radiation mitigator must be able to retain its therapeutic efficacy when administration begins 24 hours or more after exposure.

Radiation-induced changes in tissue epithelial cells, vascular capillaries and hematopoietic cells constitute basic injuries in the pathogenesis of chronic radiation damage to major organs such as heart, lung, bone marrow, liver, kidney and brain. It is important to identify novel radiation mitigators for tissue epithelial cells, capillary endothelial and hematopoietic cells for use after radiation exposure and possibly for use during radiotherapy to minimize or block normal tissue damage. It was first reported more than 50 years ago that estrogens modulate radiation sickness in animals with improved survival and accelerated recovery of hematopoiesis. Estrogens were later found to also reduce hematopoietic suppression induced by radiation therapy for cancers in the clinic. However, estrogenic toxicity at radiation mitigator doses has led to a search for safer agents. Until recent preliminary studies, it was not known if these radio-protective/-mitigator effects of estrogen were mediated by major estrogen receptor (ER) forms, ERα or ERβ. Described herein is the development of safe and effective radiological/nuclear medical countermeasures for clinical use under emergency situations, including treatments for radiation injury which are effective when administered 24 hours or later after exposure in a radiation accident or attack. Potentially, these treatments may also have future medical application for patients managed with radiotherapy administered in the clinic. Activators of a second, recently-discovered estrogen receptor, termed ER-beta, may address this need by providing a new class of radiation mitigators. Disclosed herein is modulation of ER signaling as a countermeasure to radiation injury, particularly that of the recently-discovered ERβ1, a major ERβ isoform with an intact ligand-binding domain that can be effectively targeted with small drug-like ligands. Disclosed herein, inter alia, are solutions to these and other problems in the art.

### BRIEF SUMMARY

In an aspect is provided a compound, or a pharmaceutically acceptable salt thereof, wherein the compound is selected from the group consisting of AC186, AUS131, BAY865310, 8β-VE2, 8-vinylestra-1,3,5(10)-triene-3,17β-diol, AC74131, ERB041, ERB196, Eviendep, GTx878, KB9520, Menerba, NDC1022, NDC1308, NDC1352, NDC1407, Neumune, Seala, (3S,8R,9S,10R,13S,14S,17S)-10,13-dimethyl-2,3,4,7,8,9,11,12,14,15,16,17-dodecahydro-1H-cyclopenta[a]phenanthrene-3,17-diol, (25)-7-hydroxy-2-(4-hydroxyphenyl)-2,3-dihydrochromen-4-one, (4Z)-4-(7-ethenyl-5-hydroxy-3H-1,3-benzoxazol-2-ylidene)-2-fluorocyclohexa-2,5-dien-1-one, (S)-2,3-bis(4-hydroxyphenyl)propanenitrile, and DPN (2,3-bis[4-hydroxyphenyl]-propionitrile).

In another aspect is provided a pharmaceutical composition including a pharmaceutically acceptable excipient and a compound, or pharmaceutically acceptable salt thereof, as described herein, including embodiments, including compounds described for use in a method herein or in the Compounds section above or in an example, table, figure, or claim.

In another aspect is provided a method of modulating the level of activity of estrogen receptor β, including contacting the estrogen receptor β with an effective amount of a compound, or a pharmaceutically acceptable salt thereof, as described herein, including embodiments or in any example, table, claim, or figure.

In another aspect is provided a method of treating radiation damage in a patient in need of such treatment, the method including administering a therapeutically effective amount of a compound, or a pharmaceutically acceptable salt thereof, as described herein, including embodiments or in any example, table, claim, or figure.

In another aspect is provided a method of treating cancer in a patient in need of such treatment, the method including administering a therapeutically effective amount of a compound, or a pharmaceutically acceptable salt thereof, as described herein, including embodiments or in any example, table, claim or figure.

### BRIEF DESCRIPTION OF THE DRAWINGS

FIG. 1. ERα is a ligand-activated transcription factor that mediates effects of E2; a second ER subtype, ERβ, the product of an independent gene, has sequence homology with ERα; ER's also undergo post-translational changes that affect activity (Pietras R J, Marquez-Garban D C. (2007). Clin Cancer Res 13:4672-6; Krege J H, Hodgin J B, Couse J F, Enmark E, Warner M, Mahler J F, Sar M, Korach K S, Gustafsson J A, Smithies O (1998). Proc Natl Acad Sci USA 95:15677-15682; Harris H A (2007). Mol. Endocrinol. 21(1):1-13. Epub 2006 Mar. 23. Review)).

FIG. 2. Steroid and peptide receptor signaling; cells with ER may undergo ligand-dependent receptor activation as in classical estrogen-ER signaling or ligand-independent receptor activation as mediated by growth factor receptor-induced ER phosphorylation and activation (EGFR-induced signaling); or downstream signaling by E2 due to interaction with extranuclear ER (Pietras R J, Marquez-Garban D C. (2007). Clin Cancer Res 13:4672-6).

FIG. 3. Irradiation elicits DNA damage that is either repaired to promote cell survival or results in apoptosis; estrogen signaling (ER; shown as highlights) is reported to impact these events at different nodes (see below).

FIG. 4. EGF stimulates ERβ-EGFR interactions in lung cells by 15 min; H23 lung tumor cells were cultured in phenol-red free RPMI with 1% steroid-free FBS for 48 h; in 4 experiments, cells were then treated with 2 nM EGF and collected at 0, 15, 30 min; cell lysates (2 mg) were immunoprecipitated with 1 μg of antibody EGFR (Ab-5; Calbiochem) before gel resolution, with EGFR-ERβ interactions detected by Western blot with 1 μg/ml of ERβ antibody (ABR) (Márquez D C, Lee J, Lin T, Pietras R J. Endocrine. 2001 November; 16(2):73-81); the 55 kD band corresponds to ERβ.

FIG. 5. Non-steroidal agonists for ERα or ERβ. Propylpryazoletriol (PPT) is a potent ERα agonist; while diarylpropionitrile (DPN) and ERB-041 (ERB) are highly selective in binding and activating ERβ (Harrington W R, Sheng S, Barnett D H, Petz L N, Katzenellenbogen J A, Katzenellenbogen B S. Mol Cell Endocrinol. 2003 Aug. 29; 206(1-2):13-22).

FIGS. 6A-6B. Radiation mitigation in HUVEC (FIG. 6A) by ERβ agonists DPN and ERB; and radioprotection in H23 lung tumor epithelial cells (FIG. 6B) by DPN, ERB and other estrogen agonists; using methods as before (Pietras R J, Poen J C, Gallardo D, Wongvipat P N, Lee H J, Slamon D J. Cancer Res. 1999 Mar. 15; 59(6):1347-55), cell survival curves were obtained after treatment with graded doses of ionizing radiation (RT) at a dose rate of 1 Gy/min with doses of 0, 2, 5, 10 and/or 15 Gy; HUVEC groups included cells treated with control (CON), 2 nM DPN (DPN 2) or 10 nM DPN (DPN 10) or ERB at 24 h after RT; a similar protocol was used with H23 cells using control (CON), 2 nM estrogen, 5 nM DPN or ERB or 5 nM PPT [n=3] but drugs were given 24 h before RT; after RT, cells were divided into paired dishes (n=3) and cultured 10 days (Pietras R J, Poen J C, Gallardo D, Wongvipat P N, Lee H J, Slamon D J. Cancer Res. 1999 Mar. 15; 59(6):1347-55).

FIG. 7. Radioprotection of NHBE cells by ERβ agonists DPN and ERB; cells were grown in estrogen-free media, then treated with control vehicle (CON), DPN (5 nM) or ERB (5 nM) for 2 h before irradiation (2 Gy); thereafter, cells were cultivated in vitro for up to 48 h to determine cell viability by established methods; effects of DPN and ERB on cell viability were found to be significantly different from control (P<0.001).

FIG. 8. H23 lung epithelial cells were transfected with control non-specific siRNAs (CON) or smart-pool ESR2 [ERβ] siRNA for ERβ silencing (ERbKD) as before (31,32); control cells were then treated with or without estradiol (E2) and compared with ERbKD cells treated with estradiol for 2 hrs before irradiation at 0-15 Gy as indicated in the figure (n=3 experiments); surviving cell fractions (%) are plotted on a log scale at increasing radiation doses.

FIG. 9. Estrogen and ERβ-selective ligands stimulate repair of radiation-damaged reporter DNA in H23 lung cells; a CMV-driven β-galactosidase reporter plasmid was exposed to radiation in vitro and then transfected in H23 cells (Pietras R J, Poen J C, Gallardo D, Wongvipat P N, Lee H J, Slamon D J. Cancer Res. 1999 Mar. 15; 59(6):1347-55); at 24 h after transfection, DNA repair was assayed by measuring reporter DNA expression in cells incubated with control (CON), 2 nM estrogen (E2), 0.5 nM DPN or ERB or 0.5 nM PPT beginning at the end of transfection; reporter activity is presented as % blue-stained cells in the presence of 5-bromo-4-chloro-3-indolyl-β-D-galactopyranoside, a substrate for β-galactosidase; methods were reported in detail before (Pietras R J, Poen J C, Gallardo D, Wongvipat P N, Lee H J, Slamon D J. Cancer Res. 1999 Mar. 15; 59(6):1347-55); data are from 3 experiments (mean±SEM).

FIG. 10. A549 cells (high XRCC1) were treated 2 h with control (CON) or estrogens, then irradiated at 0 or 5 Gy; cells stained with phospho-serine-1981-ATM (PS-ATM) antibody 2 h after radiation; PS-ATM was detected by anti-mouse-Alexa 488 (green); nuclear clustering of PS-ATM was induced by radiation in CON and also increased by E2 (10 nM) and DPN but not PPT (1 μM) in 3 experiments.

FIG. 11. TUNEL assay shows apoptosis in lung cells after RT; cells (E2-depleted) were treated with control (CON), E2 (10 nM), DPN (1 μM), PPT (1 μM) for 2 h followed by 15 Gy RT; after 48 h, cells were fixed, permeabilized for TUNEL assay; fluorescent green shows apoptosis (400X); TUNEL: terminal deoxynucleotidyl transferase to incorporate fluorescein-dUTP to nicked DNA of apoptotic cells.

FIG. 12. A549 lung epithelial cells were treated with control (CON), estradiol (E2; 10 nM), genistein, DPN or PPT (1 μM) for 24 h; thereafter, cell lysates were prepared for Western blots, with antibodies used to detect MDM2 (and actin loading controls).

FIG. 13. Estrogen and DPN stimulate high levels of VEGF secretion after irradiation of H23 lung epithelial cells in vitro; cells were grown in estrogen-free media, then treated with control (CON), E2 (2 nM) or DPN (5 nM) for 2 hrs before irradiation (10 Gy); VEGF secretion was then assayed by established ELISA methods after 24 hrs and expressed as percent control levels.

FIG. 14. Effects of DPN and ERB-041 given after lethal TBI in mice; mice (n=8/group) received 7.725 Gy TBI; at 24 h after irradiation, mice were treated daily for 5 days with 10 mg/kg diarylpropionitrile (DPN) subcutaneously (SC) or prinaberel (ERB-041) at 50 mg/kg by oral gavage (OG); controls received appropriate vehicle treatment (e.g. SC [CON SC] or OG [CON OG].

FIG. 15. Effects of DPN and ERB-041 given after lethal TBI in mice. Mice (n=8/group) received 8.5 Gy TBI. At 24 h after irradiation, mice were treated daily for 5 days with 10 mg/kg diarylpropionitrile (DPN) subcutaneously (SC) or prinaberel (ERB-041) at 50 mg/kg by oral gavage (OG). Controls received appropriate vehicle treatment (e.g. SC [CON SC] or OG [CON OG].

FIGS. 16A-16B. Graph FIG. 16A shows the LD50/30 values for C3H and C57Bl/6 mice and the time to “death” (FIG. 16B) using IACUC proscribed criteria for premature termination; the LD70/30 values are being used; mice are irradiated to the whole body using a Cesium source, mice are unrestrained and not anesthetized. Legend: C3H mice (left curves); C57Bl/6 mice (right curves).

FIGS. 17A-17B. FIG. 17A: PET/CT images of ¹⁸F-radiolabeled polyamides (see e.g., Harki D A, et al. (2008). Proc Natl Acad Sci USA 2008 Sep. 2; 105(35):13039-44) at 5, 30 and 120 min post-injection. From this type of data collection percent injected dose per organ over time, residency times and thus radiation dosimetry are determined (Harki D A, 2008, Id.). FIG. 17B: Biodistribution of data obtained as shown in FIG. 17A.

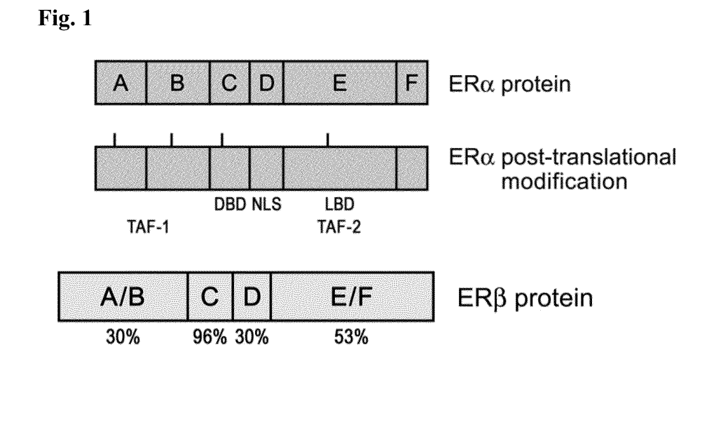

### DETAILED DESCRIPTION

Injuries to bone marrow, gastrointestinal, respiratory and cardiovascular systems are major determinants of lethality after total-body irradiation (TBI). Despite recent progress, development of other effective and safe countermeasures to structural injury and dysfunction remains urgent. Estrogens are reported to modulate radiation sickness in animals with improved survival and accelerated hematopoietic recovery after TBI. Steroid receptors, such as a second recently-discovered estrogen receptor, termed ER-beta, may provide a new class of radiation mitigators. ER-beta is expressed in most human tissues, particularly those most susceptible to TBI injury. In this study we first determined radiation mitigator effects of ER-beta-selective agonist DPN (2,3-bis[4-hydroxyphenyl]-propionitrile) in normal human lung epithelial and vascular endothelial cells in vitro.

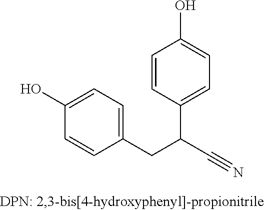 [SMILES: N#CC(Cc1ccc(O)cc1)c1ccc(O)cc1]

Cell survival is significantly improved in a dose-dependent fashion by treatment with DPN at 24-hr after irradiation as compared to controls (P<0.001). Of importance, radiation mitigator effects of DPN are abolished when lung epithelial cells are treated with inhibitory RNAs (siRNA) targeted to suppress ERβ expression by established methods (P<0.001). In addition, DPN stimulates repair of radiation-damaged reporter DNA in lung tumor epithelial cells and inhibits ATM phosphorylation at serine 1981 after irradiation. We also assessed cell death, another measurable outcome due to radiation injury, and found that DPN showed efficacy in suppressing radiation-induced apoptosis. Further, treatment with DPN at 24-hrs after irradiation (10 Gy) elicits a significant increase in VEGF secretion as assayed by ELISA methods as compared with control (P<0.001). Finally, ER-beta agonists function as radiation mitigators in animal models in vivo. Male C3H mice, 9-10 wks old, received 7.725 Gy TBI at a dose rate of 67 cGy/min. For mitigation, DPN (10 mg/kg) or vehicle was given subcutaneously daily for 5-days starting 24-hrs after TBI. DPN protected mice from lethality and improved survival when compared to controls (P<0.001). Hence, ER-beta agonists may represent a previously-unsuspected new class of radiation mitigators for use in the event of a radiation attack or accident.

Described herein are treatments for mass casualty events, with the ultimate goal of advancement of research on mitigators/treatments of short- and long-term consequences of radiation exposure for the general population in the event of a nuclear incident, accident or attack. Preliminary work on the biologic activity of a second type of estrogen receptor, ERβ, shows that agonist ligands that bind to ERβ1 isoforms have previously-unsuspected actions as radiation mitigators. In particular, diarylpropionitrile (DPN) (Harrington W R, Sheng S, Barnett D H, Petz L N, Katzenellenbogen J A, Katzenellenbogen B S. Mol Cell Endocrinol. 2003 Aug. 29; 206(1-2):13-22) is an ERβ-binding agonist with significant activity as a radiation mitigator in preclinical models. Therefore, a new class of radiation mitigators may be developed from drug-like compounds that activate ERβ1 signaling pathways that are reported to occur in constituent cells of most major organs that are injured by irradiation (Krege J H, Hodgin J B, Couse J F, Enmark E, Warner M, Mahler J F, Sar M, Korach K S, Gustafsson J A, Smithies 0 (1998). Proc Natl Acad Sci USA95:15677-15682; Harris H A (2007). Mol Endocrinol. 21(1):1-13. Epub 2006 Mar. 23. Review; Cristofaro P A, Opal S M, Palardy J E, Parejo N A, Jhung J, Keith J C Jr, Harris H A. Crit Care Med. 2006 August; 34(8):2188-93; Brush J, Lipnick S L, Phillips T, Sitko J, McDonald J T, McBride W H. Semin Radiat Oncol. 2007 April; 17(2):121-30).

Role of nuclear receptor signaling in radiation-induced tissue damage. Steroid receptors, such as the classical ERα and the recently discovered ERβ, may regulate radiation sensitivity (1-3). Activation of ERα by binding estradiol-17β (E2) is well known to elicit a conformational change in ER to allow interaction with coactivator proteins and DNA transcriptional elements.

Certain DNA repair regulators, including DNA mismatch repair gene hMSH2 and hMMS19, act as potent coactivators of ERα and ERβ (4,5). In mouse mammary epithelium, irradiation-induced DNA damage elicits increased p21/WAF1, an action markedly reduced in ovariectomized mice and restored by E2 (6). In studies on expression of steroid receptors in tissue biopsy specimens before and after radiation treatment (RT) for prostate cancer, significant upregulation of ERα and especially ERβ, but not androgen receptor, is detected in tumor cells (Torlakovic E, Lilleby W, Berner A, Torlakovic G, Chibbar R, Furre T, Fosså S D (2005). Int J Cancer. 117(3):381-6). Further, prostate tumor cells expressed no ERβ mRNA before RT, but did express ERβ transcripts 24 hrs after RT, suggesting that upregulation of ERβ after radiation may represent a protective tissue response (Torlakovic E, Lilleby W, Berner A, Torlakovic G, Chibbar R, Furre T, Fosså S D (2005). Int J Cancer. 117(3):381-6).

Estrogens exert radioprotective/radiation mitigator actions. Estrogens modulate radiation sickness in animals with improved survival and accelerated recovery of hematopoiesis (Rooks W H 2nd, Dorfman R1 (1961). Endocrinology. 68:838-43; Zhou Y, Mi M T (2005). J Radiat Res. 46(4):425-33) and also reduce hematopoietic suppression induced by RT for cancers in the clinic (Zhou Y, Mi M T (2005). J Radiat Res. 46(4):425-33). However, estrogenic toxicity at radioprotective doses has led to a search for safer agents. It is not known if these effects of E2 are mediated by ERα or ERβ. Genistein, a soy isoflavone, has structural similarity to E2 and weak affinity for binding and activating ERα but more affinity for ERβ. Treatment of sublethally-irradiated mice with genistein 24 h before RT increased survival without other significant toxicity (Zhou Y, Mi M T (2005). J Radiat Res. 46(4):425-33; Landauer M R, Srinivasan V, Seed T M (2003). J Appl Toxicol. 23(6):379-85; Davis T A, Clarke T K, Mog S R, Landauer M R (2007). Int J Radiat Biol. 83(3):141-51). However, other reports suggest that genistein at lower doses enhances radiosensitivity in prostate cancer (Raffoul J J, Wang Y, Kucuk O, Forman J D, Sarkar F H, Hillman G G (2006). BMC Cancer. 6:107). The reason for conflicting data is unclear, but, collectively, the data suggest that ERβ-directed agents may be preferred to test for modulating radiosensitivity. ERβ is widely distributed in human tissues (Krege J H, Hodgin J B, Couse J F, Enmark E, Warner M, Mahler J F, Sar M, Korach K S, Gustafsson J A, Smithies O (1998). Proc Natl Acad Sci USA95:15677-15682; Harris H A (2007). Mol Endocrinol. 21(1):1-13. Epub 2006 Mar. 23. Review; Cristofaro P A, Opal S M, Palardy J E, Parejo N A, Jhung J, Keith J C Jr, Harris H A. Crit Care Med. 2006 August; 34(8):2188-93), including many most susceptible to radiation injury (heart, vasculature, hematopoietic cells, lung, liver, kidney, brain, skin) (Brush J, Lipnick S L, Phillips T, Sitko J, McDonald J T, McBride W H. Semin Radiat Oncol. 2007 April; 17(2):121-30). Further, ERβ-selective ligands do not promote some classic E2 actions such as stimulation of uterine proliferation or ER-mediated gene expression, but these ligands do enhance survival after toxic exposures or chemotherapy-induced apoptosis in target cells (Harris H A (2007). Mol Endocrinol. 21(1):1-13. Epub 2006 Mar. 23. Review; Cristofaro P A, Opal S M, Palardy J E, Parejo N A, Jhung J, Keith J C Jr, Harris H A. Crit Care Med. 2006 August; 34(8):2188-93).

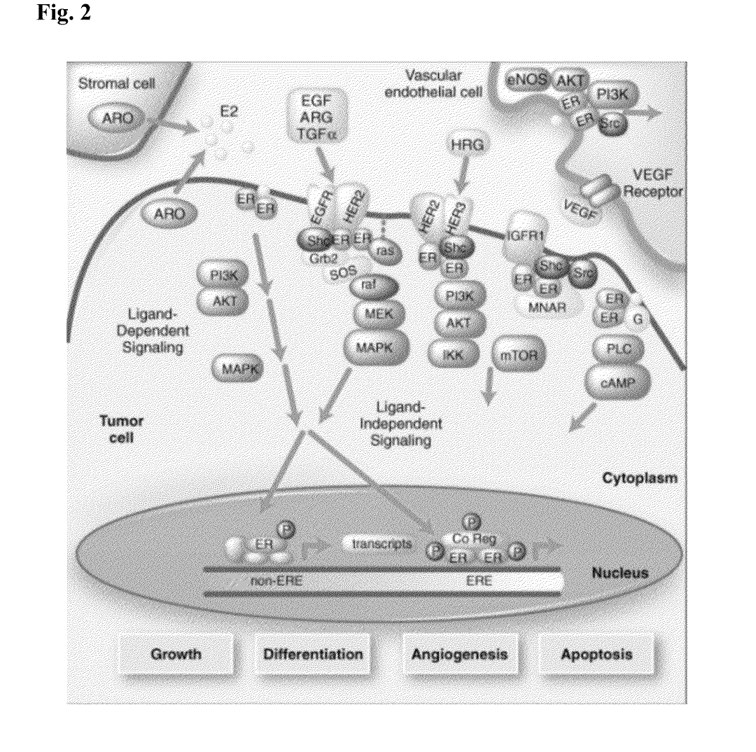

ER exerts direct nuclear actions and also regulates gene expression without direct DNA binding. The indirect genomic effects occur by protein-protein interaction with other transcription factors, such as AP-1, and with extranuclear signaling complexes that, in turn, modulate down-stream transcription (FIG. 2). Extranuclear signaling by E2, such as MAPK and AKT kinase activation, is mediated by extranuclear ER. Although extranuclear ER derives from the same transcript as nuclear ER (Razandi M, Pedram A, Greene G L, Levin E R. Mol Endocrinol. 1999 February; 13(2):307-19), extranuclear ER undergoes post-translational modification for membrane targeting (Acconcia F, Ascenzi P, Fabozzi G, Visca P, Marino M. Biochem Biophys Res Commun. 2004 Apr. 9; 316(3):878-83) and associates with other adaptor/signaling proteins, such as c-src (Pietras R J, Marquez-Garban D C. (2007). Clin Cancer Res 13:4672-6; Pietras R J, Nemere, I, Szego C M. Endocrine. 2001 April; 14(3):417-27; Song R X, Barnes C J, Zhang Z, Bao Y, Kumar R, Santen R J. Proc Natl Acad Sci USA. 2004 Feb. 17; 101(7):2076-81. Epub 2004 Feb. 5). Extranuclear ER also interacts with growth factor signaling pathways to promote cell survival (Pietras R J, Marquez-Garban D C. (2007). Clin Cancer Res 13:4672-6; Stabile L P, Davis A L, Gubish C T, Hopkins T M, Luketich J D, Christie N, Finkelstein S, Siegfried J M. Cancer Res. 2002 Apr. 1; 62(7):2141-50; Pietras R J, Márquez D C, Chen H W, Tsai E, Weinberg O, Fishbein M. Steroids. 2005 May-June; 70(5-7):372-81. Epub 2005 Mar. 25).

EGF/HER and VEGF receptors modulate radiation sensitivity, angiogenesis, apoptosis (21). Radiation-induced activation of EGFR increases cell proliferation by activating EGFR/ras/MAPK pathways, with repopulation of cells after RT (Sturla L M, Amorino G, Alexander M S, Mikkelsen R B, Valerie K, Schmidt-Ullrichr R K. J Biol Chem. 2005 Apr. 15; 280(15):14597-604. Epub 2005 February; Das A K, Chen B P, Story M D, Sato M, Minna J D, Chen D J, Nirodi C S. Cancer Res. 2007 Jun. 1; 67(11):5267-74). EGFR may also promote survival by activating PI3K/AKT kinases or by interaction with DNA-dependent protein kinase (Pietras R J, Poen J C, Gallardo D, Wongvipat P N, Lee H J, Slamon D J. Cancer Res. 1999 Mar. 15; 59(6):1347-55; Das A K, Chen B P, Story M D, Sato M, Minna J D, Chen D J, Nirodi C S. Cancer Res. 2007 Jun. 1; 67(11):5267-74). Moreover, EGFR/HER receptors regulate ligand-independent ER activation, and ER may integrate signals from growth factor pathways as well as from estrogen binding (24-26). Tissue survival after radiation injury also depends, in part, on maintenance of an adequate blood supply. Vascular endothelial growth factor (VEGF) activates endothelial signaling pathways leading to vascular proliferation and survival. VEGF production and secretion by epithelial cells is elicited by activation of ER and EGFR/HER signaling pathways in target tissues (Li D, Williams J I, Pietras R J (2002). Oncogene. 21(18):2805-14; 28. Petit A M, Rak J, Hung M C, Rockwell P, Goldstein N, Fendly B, Kerbel R S. Am J Pathol. 1997 December; 151(6): 1523-30; Pietras R J. Breast J. 2003 September-October; 9(5):361-73). Thus, promotion of ER and EGFR/HER signaling may elicit indirect cell survival effects leading to recovery from radiation injury.

In laboratory work, subcutaneous administration of the ER-beta agonist DPN (2,3-bis[4-hydroxyphenyl]-propionitrile) in mouse models in vivo exhibited potent radioprotective and radiation mitigating properties, with 100% post-irradiation survival when treatment was started 24 hours after total-body irradiation (TBI). An oral ER-beta agonist, ERB-041, exhibited similar radiation mitigating properties.

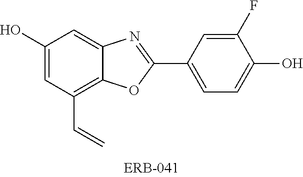 [SMILES: C=Cc1cc(O)cc2nc(-c3ccc(O)c(F)c3)oc12]

Further development of these compounds is ongoing in order to determine the maximal post-irradiation time window for administration and the response to increasing doses of TBI. DPN appears to function as a very promising radiation mitigator with a post-irradiation time window in excess of at least 24 hours. The mechanism of action likely involves in part mitigation of molecular responses to radiation-induced DNA damage and cell survival pathways in diverse body tissues that are known to harbor ER-beta signaling pathways, particularly the ER-beta1 isoform which is the only isoform with an intact ligand-binding domain for drug targeting. DPN treatment also promotes interaction of ER-beta with EGFR and increased activity of VEGF and other growth factors to promote tissue recovery. This effort has lead to development of a previously-unsuspected new class of radiation mitigators for use in the event of a radiation attack or accident. In addition, this new class of radiation mitigators/radioprotectors may also have clinical application in protecting nonmalignant tissues from injury in the course of radiation therapy for breast, prostate or other malignancies.

### DEFINITIONS

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—SO)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5 (4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ may include, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat cancer. For example certain methods herein treat cancer by decreasing a symptom of cancer. Symptoms of cancer would be known or may be determined by a person of ordinary skill in the art. The term “treating” and conjugations thereof, include prevention of an injury, pathology, condition, or disease (e.g. preventing the development of one or more symptoms of cancer). For example, certain methods herein treat radiation damage. For example certain methods herein treat radiation damage by decreasing a symptom of radiation damage. Symptoms of radiation damage would be known or may be determined by a person of ordinary skill in the art. The term “treating” and conjugations thereof, include prevention of an injury, pathology, condition, or disease (e.g. preventing the development of one or more symptoms of radiation damage).

An “effective amount” is an amount sufficient to accomplish a stated purpose (e.g. achieve the effect for which it is administered, treat a disease, reduce enzyme activity, increase enzyme activity, reduce protein function, reduce protein stability, increase protein degradation, increase protein function, increase protein stability, decrease protein degradation, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein relative to the absence of the antagonist. An “activity increasing amount,” as used herein, refers to an amount of agonist (activator) required to increase the activity of an enzyme or protein relative to the absence of the agonist. A “function disrupting amount,” as used herein, refers to the amount of antagonist (inhibitor) required to disrupt the function of an enzyme or protein relative to the absence of the antagonist. A “function increasing amount,” as used herein, refers to the amount of agonist (activator) required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. cancer, radiation damage) means that the disease (e.g. cancer, radiation damage) is caused by (in whole or in part), or a symptom of the disease is caused by (in whole or in part) the substance or substance activity or function. For example, a symptom of a disease or condition associated with an ERβ (e.g. ERβ, human ERβ, ERβ1, ERβ2, ERβ3, ERβ4, ERβ5) may be a symptom that results (entirely or partially) from an increase or decrease in the level of activity of an ERβ (e.g. ERβ, human ERβ, ERβ1, ERβ2, ERβ3, ERβ4, ERβ5). As used herein, what is described as being associated with a disease, if a causative agent, could be a target for treatment of the disease. For example, a disease associated with an increase in the level of activity of an ERβ (e.g. ERβ, human ERβ, ERβ1, ERβ2, ERβ3, ERβ4, ERβ5), may be treated with an agent (e.g. compound as described herein) effective for decreasing the level of activity of an ERβ (e.g. ERβ, human ERβ, ERβ1, ERβ2, ERβ3, ERβ4, ERβ5). For example, a disease associated with a decrease in an ERβ (e.g. ERβ, human ERβ, ERβ1, ERβ2, ERβ3, ERβ4, ERβ5), may be treated with an agent (e.g. compound as described herein) effective for increasing the level of activity of an ERβ (e.g. ERβ, human ERβ, ERβ1, ERβ2, ERβ3, ERβ4, ERβ5).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. a component of an ERβ (e.g. ERβ, human ERβ, ERβ1, ERβ2, ERβ3, ERβ4, ERβ5) protein pathway or an ERβ (e.g. ERβ, human ERβ, ERβ1, ERβ2, ERβ3, ERβ4, ERβ5)). In some embodiments contacting includes allowing a compound described herein to interact with a protein or enzyme that is involved in a signaling pathway.

As defined herein, the term “inhibition”, “inhibit”, “inhibiting” and the like in reference to a protein-inhibitor (e.g. antagonist) interaction means negatively affecting (e.g. decreasing) the level of activity or function of the protein (e.g. an ERβ (e.g. ERβ, human ERβ, ERβ1, ERβ2, ERβ3, ERβ4, ERβ5)) relative to the level of activity or function of the protein (e.g. an ERβ (e.g. ERβ, human ERβ, ERβ1, ERβ2, ERβ3, ERβ4, ERβ5)) in the absence of the inhibitor. In some embodiments inhibition refers to reduction of a disease or symptoms of disease (e.g. cancer or radiation damage). In some embodiments, inhibition refers to a reduction in the level of activity of a signal transduction pathway or signaling pathway (e.g. an ERβ (e.g. ERβ, human ERβ, ERβ1, ERβ2, ERβ3, ERβ4, ERβ5) protein pathway). Thus, inhibition may include, at least in part, partially or totally blocking stimulation, decreasing, preventing, or delaying activation, or inactivating, desensitizing, or down-regulating signal transduction or enzymatic activity or the amount of a protein (e.g. an ERβ (e.g. ERβ, human ERβ, ERβ1, ERβ2, ERβ3, ERβ4, ERβ5)). Inhibition may include, at least in part, partially or totally decreasing stimulation, decreasing activation, or deactivating, desensitizing, or down-regulating signal transduction or enzymatic activity or the amount of a protein (e.g. an ERβ (e.g. ERβ, human ERβ, ERβ1, ERβ2, ERβ3, ERβ4, ERβ5)) that may modulate the level of another protein or modulate cell survival (e.g. decreasing the level of activity of a component of an ERβ (e.g. ERβ, human ERβ, ERβ1, ERβ2, ERβ3, ERβ4, ERβ5) protein pathway may decrease cancer cell survival or radiation damage in cells that may or may not have an increase in the level of activity of a component of an ERβ (e.g. ERβ, human ERβ, ERβ1, ERβ2, ERβ3, ERβ4, ERβ5) protein pathway relative to a non-disease control).

As defined herein, the terms “activation”, “activate”, “activating” and the like in reference to a protein-activator (e.g. agonist) interaction means positively affecting (e.g. increasing) the activity or function of the protein (e.g. an ERβ (e.g. ERβ, human ERβ, ERβ1, ERβ2, ERβ3, ERβ4, ERβ5)) relative to the activity or function of the protein (e.g. an ERβ (e.g. ERβ, human ERβ, ERβ1, ERβ2, ERβ3, ERβ4, ERβ5)) in the absence of the activator (e.g. compound described herein). In some embodiments, activation refers to an increase in the activity of a signal transduction pathway or signaling pathway (e.g. an ERβ (e.g. ERβ, human ERβ, ERβ1, ERβ2, ERβ3, ERβ4, ERβ5) protein pathway). Thus, activation may include, at least in part, partially or totally increasing stimulation, increasing or enabling activation, or activating, sensitizing, or up-regulating signal transduction or enzymatic activity or the amount of a protein (e.g. an ERβ (e.g. ERβ, human ERβ, ERβ1, ERβ2, ERβ3, ERβ4, ERβ5)) decreased in a disease (e.g. level of a component of an ERβ (e.g. ERβ, human ERβ, ERβ1, ERβ2, ERβ3, ERβ4, ERβ5)) protein pathway associated with cancer or radiation damage). Activation may include, at least in part, partially or totally increasing stimulation, increasing or enabling activation, or activating, sensitizing, or up-regulating signal transduction or enzymatic activity or the amount of a protein (e.g. an ERβ (e.g. ERβ, human ERβ, ERβ1, ERβ2, ERβ3, ERβ4, ERβ5)) protein pathway) that may modulate the level of another protein or modulate cell survival (e.g. increasing the level of activity of a component of an ERβ (e.g. ERβ, human ERβ, ERβ1, ERβ2, ERβ3, ERβ4, ERβ5) protein pathway may decrease cancer cell survival or radiation damage in cells that may or may not have a reduction in the level of activity of a component of an ERβ (e.g. ERβ, human ERβ, ERβ1, ERβ2, ERβ3, ERβ4, ERβ5) protein pathway relative to a non-disease control).

The term “modulator” refers to a composition that increases or decreases the level of a target molecule or the function of a target molecule. In some embodiments, a modulator of a component of an ERβ (e.g. ERβ, human ERβ, ERβ1, ERβ2, ERβ3, ERβ4, ERβ5) protein pathway (e.g. an ERβ (e.g. ERβ, human ERβ, ERβ1, ERβ2, ERβ3, ERβ4, ERβ5)) is a compound that reduces the severity of one or more symptoms of a disease associated with a component of an ERβ (e.g. ERβ, human ERβ, ERβ1, ERβ2, ERβ3, ERβ4, ERβ5) protein pathway (e.g. disease associated with an increase of the level of activity or amount of a component of an ERβ (e.g. ERβ, human ERβ, ERβ1, ERβ2, ERβ3, ERβ4, ERβ5)) protein pathway (e.g. an ERβ (e.g. ERβ, human ERβ, ERβ1, ERβ2, ERβ3, ERβ4, ERβ5)), for example cancer or radiation damage) or a disease that is not caused by a component of an ERβ (e.g. ERβ, human ERβ, ERβ1, ERβ2, ERβ3, ERβ4, ERβ5) pathway but may benefit from modulation of the level of activity or amount of a component of an ERβ (e.g. ERβ, human ERβ, ERβ1, ERβ2, ERβ3, ERβ4, ERβ5) protein pathway (e.g. an ERβ (e.g. ERβ, human ERβ, ERβ1, ERβ2, ERβ3, ERβ4, ERβ5)). In embodiments, a modulator of the level of activity or amount of a component of an ERβ (e.g. ERβ, human ERβ, ERβ1, ERβ2, ERβ3, ERβ4, ERβ5) protein pathway (e.g. an ERβ (e.g. ERβ, human ERβ, ERβ1, ERβ2, ERβ3, ERβ4, ERβ5)) is an anti-cancer agent or a radiation mitigator or radiation protector.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin P E (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. Included in anti-cancer agents are conventional radiotherapeutic agents including, but not limited to radionuclides such as ⁴⁷Sc, ⁶⁴Cu, ⁶⁷Cu, ⁸⁹Sr, ⁸⁶Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies.

“Chemotherapeutic” or “chemotherapeutic agent” is used in accordance with its plain ordinary meaning and refers to a chemical composition or compound having antineoplastic properties or the ability to inhibit the growth or proliferation of cells.

“Patient” or “subject in need thereof” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In some embodiments, a subject is human.

“Disease” or “condition” refer to a state of being or health status of a patient or subject capable of being treated with a compound, pharmaceutical composition, or method provided herein. In some embodiments, the disease is radiation damage. In some embodiments, the disease is radiation damage associated with anti-cancer treatment. In some embodiments, the disease is associated with radiation exposure. In some embodiments, the disease is radiation poisoning. In some embodiments, the disease is exposure to radiation. In some embodiments, the disease is a disease related to (e.g. caused by) a decrease in the level (e.g. of activity or protein) of a component of an ERβ (e.g. ERβ, human ERβ, ERβ1, ERβ2, ERβ3, ERβ4, ERβ5)) protein pathway (e.g. an ERβ (e.g. ERβ, human ERβ, ERβ1, ERβ2, ERβ3, ERβ4, ERβ5)). In some embodiments, the disease is a disease related to (e.g. caused by) a decrease in the level (e.g. of activity or protein) of an ERβ (e.g. ERβ, human ERβ, ERβ1, ERβ2, ERβ3, ERβ4, ERβ5)) protein. In some embodiments, the disease is a cancer associated with a decrease in the level (e.g. level of activity or amount) of an ERβ (e.g. ERβ, human ERβ, ERβ1, ERβ2, ERβ3, ERβ4, ERβ5)) protein. In some embodiments, the disease is a cancer associated with a decrease in the level (e.g. level of activity or amount) of a component of an ERβ (e.g. ERβ, human ERβ, ERβ1, ERβ2, ERβ3, ERβ4, ERβ5)) protein pathway (e.g. an ERβ (e.g. ERβ, human ERβ, ERβ1, ERβ2, ERβ3, ERβ4, ERβ5)). In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples may include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means administration by any route, including but not limited to oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, radiation). The compound of the invention can be administered alone or can be coadministered to the patient. Coadministration is meant to include simultaneous or sequential administration of the compound individually or in combination (more than one compound or agent or with radiation). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. an ERβ (e.g. ERβ, human ERβ, ERβ1, ERβ2, ERβ3, ERβ4, ERβ5)), and/or reducing, eliminating, or slowing the progression of disease symptoms (e.g. symptoms of cancer or radiation damage). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer, radiation damage, symptom of radiation damage), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer or radiation damage, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours or more of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer such as radiation or surgery. In some embodiments, the compounds described herein may be combined with treatments for radiation damage.

The term “ERβ” or “estrogen receptor β” are used interchangeably and refer to the protein “estrogen receptor beta”. In embodiments, ERβ refers to the human protein ERβ. Included in the term ERβ are the wildtype and mutant forms of the protein. In embodiments, ERβ refers to the protein associated with Entrez Gene 2100, OMIM 601663, UniProt Q92731, and/or RefSeq (protein) NP_{—}001035365. In embodiments, ERβ refers to the protein associated with one or more of the database entries listed immediately above at the time of filing of the present application. In embodiments, ERβ refers to splice variant ERβ1. In embodiments, ERβ refers to splice variant ERβ2. In embodiments, ERβ refers to splice variant ERβ3. In embodiments, ERβ refers to splice variant ERβ4. In embodiments, ERβ refers to splice variant ERβ5. In embodiments, ERβ refers to the wildtype human protein ERβ.

The term “ERβ family protein pathway” refers to a signal transduction pathway including an ERβ protein. In embodiments an ERβ family protein pathway is an ERβ1 protein pathway. In embodiments an ERβ family protein pathway is an ERβ2 protein pathway. In embodiments an ERβ family protein pathway is an ERβ3 protein pathway. In embodiments an ERβ family protein pathway is an ERβ4 protein pathway. In embodiments an ERβ family protein pathway is an ERβ5 protein pathway. A component of an ERβ family protein pathway refers to a protein included in a signal transduction pathway including an ERβ protein.

The term “radiation mitigator” is used in accordance with its plain ordinary meaning and refers to an agent or composition capable of reducing or treating radiation damage, including but not limited to one or more symptoms of radiation damage. Examples of radiation mitigators include but are not limited to growth factors (e.g. palifermin), protease inhibitors (Bowman-Birk proteinase inhibitor), dithiolthione (e.g. oltipraz), ACE inhibitors (e.g. captopril, enalapril, ramipril), isoflavone (e.g. genistein), Hmg-CoA reductase inhibitors (e.g. simvastatin, pravastatin, lovastatin), COX2 inhibitors/NSAIDS (e.g. celecoxib, aspirin, ibuprofen), TGF-beta signaling inhibitors (e.g. halofuginone, 1D11, SM16).

The terms “radiation protector” or “radioprotector” or “radiation protectant” are used in accordance with their plain ordinary meaning and refer to an agent or composition capable of preventing (in whole or in part) radiation damage, including but not limited to one or more symptoms of radiation damage.

The term “radiation damage” is used according to it plain ordinary meaning and refers to toxicity, structural damage, impaired function, or other negative effect on a subject (e.g. patient, organ, tissue, cell, biological sample, sub-cellular component or structure, nucleic acid, protein, or other biological composition), wherein the toxicity, structural damage, impaired function, or other negative effect is associated with exposure of the composition to radiation (e.g. ionizing radiation, nuclear material exposure, nuclear attack, nuclear accident, radiological attack, radiological accident, radiological material exposure, radiation therapy, external beam radiation therapy, conventional external beam radiation therapy, stereotactic radiation, stereotactic radiosurgery, stereotactic body radiation therapy, virtual simulation, 3-dimensional conformal radiation therapy, intensity-modulated radiation therapy, particle therapy, proton therapy, brachytherapy, or radioisotope therapy).). In embodiments, the radiation damage is the damage due to toxic effects of radiation.

A “detectable moiety” is a composition detectable by spectroscopic, photochemical, biochemical, immunochemical, chemical, magnetic resonance imaging, or other physical means. For example, useful detectable moieties include ³²P, fluorescent dyes, electron-dense reagents, biotin, digoxigenin, paramagnetic molecules, superparamagnetic iron oxide, monochrystalline iron oxide, Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, iodinated contrast agents (e.g. iohexyl, iodixanol, ioversol, iopamidol, ioxilan, iopromide, diatrizoate, metrizoate, ioxaglate), barium sulfate, thorium dioxide, gold, fluorophores, two-photon fluorophores, or radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, or ²¹²Bi.

Compounds

In an aspect is provided a compound, or a pharmaceutically acceptable salt thereof, wherein the compound is selected from the group consisting of AC186, AUS131, BAY865310, 8β-VE2,8-vinylestra-1,3,5(10)-triene-3,17β-diol, AC74131, ERB041, ERB196, Eviendep, GTx878, KB9520, Menerba, NDC1022, NDC1308, NDC1352, NDC1407, Neumune, Seala, (3S,8R,9S,10R,13S,14S,17S)-10,13-dimethyl-2,3,4,7,8,9,11,12,14,15,16,17-dodecahydro-1H-cyclopenta[a]phenanthrene-3,17-diol, (2S)-7-hydroxy-2-(4-hydroxyphenyl)-2,3-dihydrochromen-4-one, (4Z)-4-(7-ethenyl-5-hydroxy-3H-1,3-benzoxazol-2-ylidene)-2-fluorocyclohexa-2,5-dien-1-one, (S)-2,3-bis(4-hydroxyphenyl)propanenitrile, and DPN (2,3-bis[4-hydroxyphenyl]-propionitrile).

In embodiments of the compound, or a pharmaceutically acceptable salt thereof, the compound is AC186. In embodiments of the compound, or a pharmaceutically acceptable salt thereof, the compound is AUS131. In embodiments of the compound, or a pharmaceutically acceptable salt thereof, the compound is BAY865310. In embodiments of the compound, or a pharmaceutically acceptable salt thereof, the compound is 8β-VE2. In embodiments of the compound, or a pharmaceutically acceptable salt thereof, the compound is 8-vinylestra-1,3,5(10)-triene-3,17β-diol. In embodiments of the compound, or a pharmaceutically acceptable salt thereof, the compound is AC74131. In embodiments of the compound, or a pharmaceutically acceptable salt thereof, the compound is ERB041. In embodiments of the compound, or a pharmaceutically acceptable salt thereof, the compound is ERB196. In embodiments of the compound, or a pharmaceutically acceptable salt thereof, the compound is Eviendep. In embodiments of the compound, or a pharmaceutically acceptable salt thereof, the compound is GTx878. In embodiments of the compound, or a pharmaceutically acceptable salt thereof, the compound is KB9520. In embodiments of the compound, or a pharmaceutically acceptable salt thereof, the compound is Menerba. In embodiments of the compound, or a pharmaceutically acceptable salt thereof, the compound is NDC1022. In embodiments of the compound, or a pharmaceutically acceptable salt thereof, the compound is NDC1308. In embodiments of the compound, or a pharmaceutically acceptable salt thereof, the compound is NDC1352. In embodiments of the compound, or a pharmaceutically acceptable salt thereof, the compound is NDC1407. In embodiments of the compound, or a pharmaceutically acceptable salt thereof, the compound is Neumune. In embodiments of the compound, or a pharmaceutically acceptable salt thereof, the compound is Seala. In embodiments of the compound, or a pharmaceutically acceptable salt thereof, the compound is (3S,8R,9S,10R,13S,14S,17S)-10,13-dimethyl-2,3,4,7,8,9,11,12,14,15,16,17-dodecahydro-1H-cyclopenta[a]phenanthrene-3,17-diol. In embodiments of the compound, or a pharmaceutically acceptable salt thereof, the compound is (2S)-7-hydroxy-2-(4-hydroxyphenyl)-2,3-dihydrochromen-4-one. In embodiments of the compound, or a pharmaceutically acceptable salt thereof, the compound is (4Z)-4-(7-ethenyl-5-hydroxy-3H-1,3-benzoxazol-2-ylidene)-2-fluorocyclohexa-2,5-dien-1-one. In embodiments of the compound, or a pharmaceutically acceptable salt thereof, the compound is (S)-2,3-bis(4-hydroxyphenyl)propanenitrile. In embodiments of the compound, or a pharmaceutically acceptable salt thereof, the compound is DPN (2,3-bis[4-hydroxyphenyl]-propionitrile).

In embodiments, the compound is in a pharmaceutical composition including a pharmaceutically acceptable excipient. In embodiments, the compound is in a pharmaceutically acceptable salt. In embodiments, the compound is co-administered with a second agent (e.g. therapeutic agent, anti-cancer agent, radiation, radioactive material, radiation damage therapy, radiation mitigator, or radiation protector). In embodiments, the second agent is administered in a therapeutically effective amount. In embodiments, the compound and a second agent (e.g. therapeutic agent) are in a pharmaceutical composition including a pharmaceutically acceptable excipient. In embodiments, the second agent is a radiation mitigator. In embodiments, the second agent is a radiation protector. In embodiments, the second agent is an estrogen receptor β agonist. In embodiments, the second agent is a radioactive material. In embodiments, the second agent is radiation (e.g. ionizing radiation). In embodiments, the second agent is an anti-cancer agent. In embodiments, the second agent is a chemotherapeutic. In embodiments, the second agent is a radiation damage therapy.

In embodiments, the compound is a compound described herein. In embodiments, the compound is a compound described in the Examples, an example, a table, Table 1, Table 2, Table 3, Table 4, the figures, a figure, or a claim, each included herein. In embodiments, the compound is a compound described in the method sections herein below.

Pharmaceutical Compositions

In another aspect is provided a pharmaceutical composition including a pharmaceutically acceptable excipient and a compound, or pharmaceutically acceptable salt thereof, as described herein, including embodiments, including compounds described for use in a method herein or in the Compounds section above or in an example, table, figure, or claim.

In embodiments of the pharmaceutical compositions, the pharmaceutical composition includes a compound, or pharmaceutically acceptable salt thereof, as described herein in a therapeutically effective amount. In embodiments of the pharmaceutical compositions, the pharmaceutical composition includes a second agent (e.g. therapeutic agent, anti-cancer agent, radiation, radioactive material, radiation damage therapy, radiation mitigator, or radiation protector). In embodiments of the pharmaceutical compositions, the pharmaceutical composition includes a second agent in a therapeutically effective amount. In embodiments of the pharmaceutical compositions, the second agent is an agent for treating cancer. In embodiments of the pharmaceutical compositions, the second agent is a radioactive material. In embodiments of the pharmaceutical compositions, the second agent is radiation. In embodiments of the pharmaceutical compositions, the second agent is a radiation mitigator. In embodiments of the pharmaceutical compositions, the second agent is radiation protector. In embodiments of the pharmaceutical compositions, the second agent is radiation damage therapy.

Methods of Modulating Activity

In another aspect is provided a method of modulating the level of activity of estrogen receptor β, including contacting the estrogen receptor β with an effective amount of a compound, or a pharmaceutically acceptable salt thereof, as described herein, including embodiments or in any example, table, claim, or figure.

In embodiments, the method of modulating is a method of inhibiting. In embodiments, the method of modulating is a method of increasing. In embodiments, the estrogen receptor β is in vitro. In embodiments, the estrogen receptor β is in a subject. In embodiments, the activity is estrogen receptor β enzymatic activity. In embodiments, the activity is estrogen receptor β protein binding activity. In embodiments, the estrogen receptor β protein binding activity is estrogen receptor β-EGFR protein binding activity. In embodiments, the activity is increasing the level of VEGF protein or activity. In embodiments, the activity of estrogen receptor β is increasing cell survival. In embodiments, the activity of estrogen receptor β is increasing survival of lung epithelial cells. In embodiments, the activity of estrogen receptor β is increasing survival of vascular endothelial cells. In embodiments, the activity of estrogen receptor β is inhibiting apoptosis. In embodiments, the activity of estrogen receptor β is increasing VEGF secretion. In embodiments, the activity of estrogen receptor β is decreasing cell death. In embodiments, the activity of estrogen receptor β is decreasing subject death. In embodiments, the activity of estrogen receptor β is increasing recovery of hematopoiesis. In embodiments, the activity of estrogen receptor β is decreasing hematopoiesis suppression. In embodiments, the activity of estrogen receptor β is increasing DNA repair. In embodiments, the activity of estrogen receptor β is decreasing DNA damage. In embodiments, the compound is in a pharmaceutical composition including a pharmaceutically acceptable excipient. In embodiments, the compound is in a pharmaceutically acceptable salt. In embodiments of the method, the compound is co-administered with a second agent (e.g. therapeutic agent, anti-cancer agent, radiation, radioactive material, radiation damage therapy, radiation mitigator, or radiation protector). In embodiments of the method, the second agent is administered in a therapeutically effective amount.

Methods of Treatment

In another aspect is provided a method of treating radiation damage in a patient in need of such treatment, the method including administering a therapeutically effective amount of a compound, or a pharmaceutically acceptable salt thereof, as described herein, including embodiments or in any example, table, claim, or figure.

In embodiments, the compound, or a pharmaceutically acceptable salt thereof, is an estrogen receptor β agonist. In embodiments, the compound, or a pharmaceutically acceptable salt thereof, is a radiation mitigator. In embodiments, the compound, or a pharmaceutically acceptable salt thereof, is a radiation protector. In embodiments, treating is preventing radiation damage (in whole or in part). In embodiments, the radiation damage is associated with an anti-cancer treatment (e.g. radiation therapy). In embodiments, the radiation damage is associated with exposure to nuclear material (e.g. nuclear attack, nuclear accident or spill). In embodiments, the radiation damage is associated with exposure to radiological material (e.g. radiological attack, radiological accident or spill). In embodiments, the radiation damage is caused by ionizing radiation. In embodiments, the radiation damage is to the bone marrow. In embodiments, the radiation damage is to the gastrointestinal tract. In embodiments, the radiation damage is to the respiratory system (e.g. lungs). In embodiments, the radiation damage is to the cardiovascular system. In embodiments, the radiation damage is to epithelial cells. In embodiments, the radiation damage is to vascular capillaries. In embodiments, the radiation damage is to hematopoietic cells. In embodiments, the radiation damage is to the heart. In embodiments, the radiation damage is to the lungs. In embodiments, the radiation damage is to bone marrow. In embodiments, the radiation damage is to the liver. In embodiments, the radiation damage is to the kidney. In embodiments, the radiation damage is to the brain. In embodiments, the radiation damage is to the vasculature. In embodiments, the radiation damage is to the skin. In embodiments, the radiation damage is to DNA. In embodiments, the compound, or a pharmaceutically acceptable salt thereof, as described herein, including embodiments or in any example, table, claim, or figure is co-administered with radiation therapy (e.g. cancer therapy). In embodiments, the compound, or a pharmaceutically acceptable salt thereof, as described herein, including embodiments or in any example, table, claim, or figure is administered to the patient about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 144, 168, 192, 216, or 240 hours after exposure to a source of radiation damage. In embodiments, the compound, or a pharmaceutically acceptable salt thereof, as described herein, including embodiments or in any example, table, claim, or figure is administered to the patient 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 144, 168, 192, 216, or 240 hours after exposure to a source of radiation damage. In embodiments, the compound, or a pharmaceutically acceptable salt thereof, as described herein, including embodiments or in any example, table, claim, or figure is administered to the patient after more than about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 144, 168, 192, 216, or 240 hours following exposure to a source of radiation damage. In embodiments, the compound, or a pharmaceutically acceptable salt thereof, as described herein, including embodiments or in any example, table, claim, or figure is administered to the patient after more than 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 144, 168, 192, 216, or 240 hours following exposure to a source of radiation damage.

In embodiments, the radiation damage is associated with radiation therapy. In embodiments, the radiation therapy is selected from the group consisting of external beam radiation therapy, conventional external beam radiation therapy, stereotactic radiation, stereotactic radiosurgery, stereotactic body radiation therapy, virtual simulation, 3-dimensional conformal radiation therapy, intensity-modulated radiation therapy, particle therapy, proton therapy, brachytherapy, and radioisotope therapy. In embodiments, the compound, or a pharmaceutically acceptable salt thereof, as described herein, including embodiments or in any example, table, claim, or figure is co-administered (e.g. within about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 144, 168, 192, 216, or 240 hours; or within 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 144, 168, 192, 216, or 240 hours) to the patient with radiation therapy (e.g. external beam radiation therapy, conventional external beam radiation therapy, stereotactic radiation, stereotactic radiosurgery, stereotactic body radiation therapy, virtual simulation, 3-dimensional conformal radiation therapy, intensity-modulated radiation therapy, particle therapy, proton therapy, brachytherapy, or radioisotope therapy).

In embodiments, the radiation damage symptom treated is selected from the group consisting of nausea, vomiting, damage to epithelial surfaces (e.g. red or itchy skin at the site of radiation), mouth sores, throat sores, stomach sores, intestinal discomfort (e.g. diarrhea, pain, nausea), edema, swelling, infertility, fibrosis, epilation, dryness (e.g. xerostomia, xerophthalmia), lymphedema, secondary malignancy (cancer), heart disease, cognitive decline, hematopoiesis suppression, radiation proctitis, hair loss, heart damage, vasculature damage, hematopoietic cell damage, lung damage, liver damage, kidney damage, brain damage, skin damage, leucopenia (low white blood cells), and death.

In another aspect is provided a method of treating cancer in a patient in need of such treatment, the method including administering a therapeutically effective amount of a compound, or a pharmaceutically acceptable salt thereof, as described herein, including embodiments or in any example, table, claim, or figure. In embodiments, the cancer is breast cancer. In embodiments, the cancer is ERα-positive breast cancer.

In embodiments, the compound is in a pharmaceutical composition including a pharmaceutically acceptable excipient. In embodiments, the compound is in a pharmaceutically acceptable salt. In embodiments of the method, the compound is co-administered with a second agent (e.g. therapeutic agent, anti-cancer agent, radiation, radioactive material, radiation damage therapy, radiation mitigator, or radiation protector). In embodiments of the method, the second agent is administered in a therapeutically effective amount. In embodiments, the second agent is an anti-cancer agent (e.g. radioactive material, radiation). Suitable anti-cancer agents for co-administration in a method described herein may be determined by one of ordinary skill in the art.

### EXAMPLES

Injuries to bone marrow, gastrointestinal tract, respiratory and cardiovascular systems are major determinants of lethality after total-body irradiation (TBI). Although some progress has been made in the management of systemic radiation injury, development of additional effective and safe countermeasures against structural injury and dysfunction remain an urgent need, especially in view of increasing risks of nuclear or radiological accidents or attacks. To be useful in an actual mass casualty situation, a medial radiation mitigator must be able to retain its therapeutic efficacy when administration begins 24 hours or more after exposure. Activators of a second, recently-discovered estrogen receptor, termed ERβ, may address this need by providing a new class of radiation mitigators. In preliminary work, subcutaneous administration of the ERβ agonist DPN (2,3-bis[4-hydroxyphenyl]-propionitrile) in mouse models in vivo exhibited potent radioprotective and radiation mitigating properties, with 100% post-irradiation survival when treatment was started 24 hours after TBI. Described herein is the development of medical countermeasures to enhance survival in irradiated animal models that are predictive of human responses. Compounds can be administered as a single- or multiple-dose after radiation exposure with administration of the first dose beginning at a minimum of 24 hours or later post-irradiation. Research emphasis can be on broad multi-organ activity. The countermeasures development is targeted to mitigate or treat radiation damage that leads to mortality and/or major morbidities, including acute and/or delayed radiation syndromes encompassing radiation injury to hematopoietic, gastrointestinal, cutaneous, pulmonary, renal, cardiovascular and/or central nervous system compartments of the body. Male C3H and C57BI/6 mice can be exposed to 7.725-9.5 Gy TBI; DPN or control vehicle can be given by daily subcutaneous injections for varying numbers of days, beginning 24-72 h after irradiation, and 30-day animal survival can be recorded. The contribution of selected systemic cytokine levels can be estimated by analyzing plasma samples obtained from the carotid artery, and tissue specimens can be assessed postmortem for histologic and immunohistochemical indices of molecular responses to radiation injury. Based on work to date, DPN appears to function as a very promising radiation mitigator with a post-irradiation time window in excess of at least 24 hours. The mechanism of action likely involves in part mitigation of molecular responses to radiation-induced DNA damage and cell survival pathways in diverse body tissues that are known to harbor ERβ signaling pathways, particularly the ERβ1 isoform which is the only isoform with an intact ligand-binding domain for drug targeting. DPN treatment also promotes interaction of ERβ with EGFR and increased activity of VEGF and other growth factors to promote tissue recovery. This multidisciplinary effort can lead to development of a previously-unsuspected new class of radiation mitigators for use in the event of a radiation attack or accident.

in vivo microPET, microCT, optical bioluminescent, fluorescent imaging and ex-vivo autoradiography. The center has commercial microPET systems (P4: 2000-2007, Focus 220: 2003, Inveon DPET: 2008), optical imaging systems (3 IVIS 100 systems replaced by 2 Luminas and a CRi Maestro), along with autoradiography and cryostat systems. For radiochemistry, the facility has 3 custom designed manual synthesis modules that can create probes requiring up to 3 separate reaction vessels. An automated multi-pot radiochemistry system called ELIXYS, now commercially available, can reproducibly create probes using disposable reagent cassettes to enable fast turnover to make multiple probes or synthesis runs. the clinical cyclotron/radiochemistry facility can provide ¹⁸F.

### 1. IDENTIFY ESTROGEN RECEPTOR (ER)-SELECTIVE LIGANDS TO PROMOTE SURVIVAL AND PROTECT CRITICAL TISSUES POST-RADIATION

Preliminary findings suggest that agonists to ERβ1 are critical in the response to radiation injury. Hence, a major emphasis of this project is characterization of molecular pathways underlying activation of this receptor after irradiation. Experiments include the following: a) Assess expression and radiation mitigator activity of ERβ and ERα in human lung and colon epithelial cells and vascular endothelial cells after irradiation in vitro. ER expression is assayed before and after irradiation and in cells treated with and without ERα- and ERβ-selective ligands administered at selected times and schedules after irradiation. b) Downstream signaling to cell cycle arrest, DNA repair and survival pathways can be correlated with treatments. c) ER signaling in cells after irradiation using ER siRNA's to selectively suppress ERα or ERβ.expression can further help to decipher specific receptor functions.

### 2. DEVELOP ER-SELECTIVE LIGANDS AS RADIATION MITIGATORS ADMINISTERED AFTER RADIATION EXPOSURE

Develop ER-selective ligands as radiation mitigators administered after radiation exposure, with an emphasis on broad activity to reduce major morbidity and promote survival using animal models. Promising radiation mitigators detected in vitro can be used for in vivo testing in mice exposed to lethal irradiation. Based on preliminary findings, ERβ1 agonist DPN is a primary focus of these studies of drug (e.g. DPN, ERB-041) doses, routes of administration and schedules at 24, 48 and 72 hours post-irradiation, and activity in response to increasing radiation exposures can be performed in vivo using C3H and C57BI/6 mouse models.

Studies of candidate drug biodistribution and potential pharmacokinetic properties can be performed in vivo.

### 3. ER-SELECTIVE LIGANDS INCREASE SURVIVAL OF EPITHELIAL AND ENDOTHELIAL CELLS POST-RADIATION

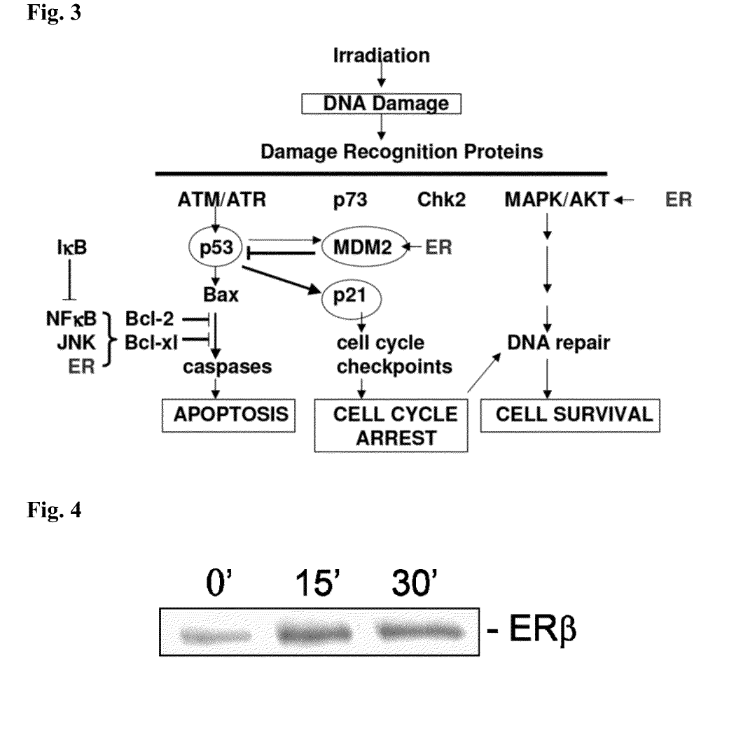

It is important to identify novel radiation mitigators for tissue epithelial, capillary endothelial and hematopoietic cells for use after radiation exposure. We can focus on evaluating the activity of nuclear receptor signaling, particularly that of the recently-discovered ER-beta-1 (ERβ1), as a countermeasure to radiation injury. The results of preliminary studies are presented first, followed by an outline of further experiments. We can focus on actions of ERβ-selective ligands administered at selected times post-irradiation in cell models in vitro and animal models in vivo in response to radiation, including modulation of apoptosis, DNA repair, cell cycle (e.g. G2/M arrest) and induction of growth factors for cell survival (see FIG. 3). An overall scheme to perform studies of ER interaction with radiation-sensing and effector proteins is presented in FIG. 3. On the basis of independent reports, estrogen receptor signaling is known to impact the cellular response to radiation injury at several potential nodes in affected cells (FIG. 3).

EGF stimulates ERβ-EGFR interaction in epithelial cells. To assess the idea that ERβ interacts with EGFR to promote cross-talk in signaling, the association of these molecules in co-immunoprecipitation studies was tested in vitro (Márquez D C, Lee J, Lin T, Pietras R J. Endocrine. 2001 November; 16(2):73-81) (FIG. 4). Results show increased ERβ association with EGFR after EGF treatment and support the notion that ERβ may cross-talk with EGFR for downstream signaling.

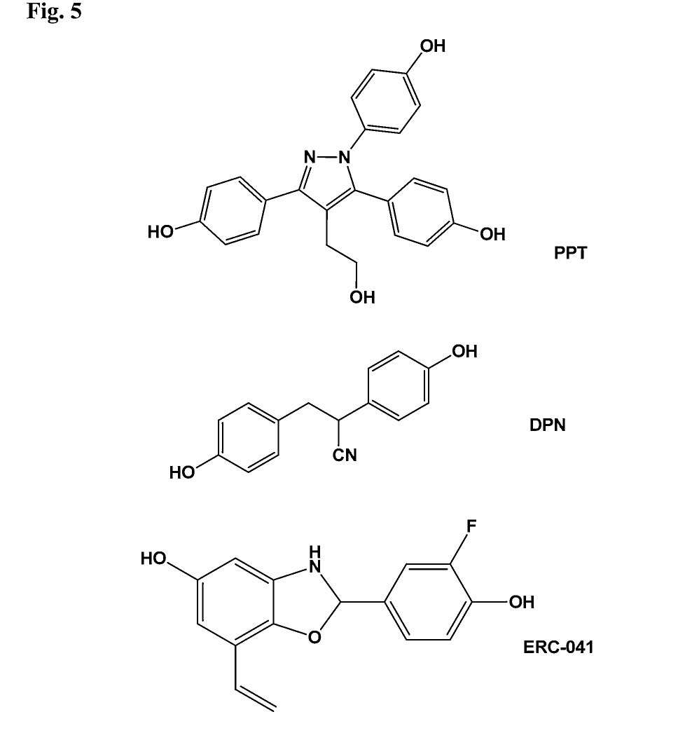

ER-selective ligands increase survival of epithelial and endothelial cells post-radiation. To assess potency of ERα- and ERβ-selective agonists (FIG. 5) in promoting cell survival after irradiation, H23 lung epithelial cells (express ERβ and ERα) and ERβ-positive human vascular endothelial cells (HUVEC) were irradiated with and without treatment with DPN or ERB-041, highly selective ERβ agonists, or known ERα-selective agonist PPT (Harris H A (2007). Mol Endocrinol. 21(1):1-13. Epub 2006 Mar. 23. Review; Harrington W R, Sheng S, Barnett D H, Petz L N, Katzenellenbogen J A, Katzenellenbogen B S. Mol Cell Endocrinol. 2003 Aug. 29; 206(1-2):13-22).

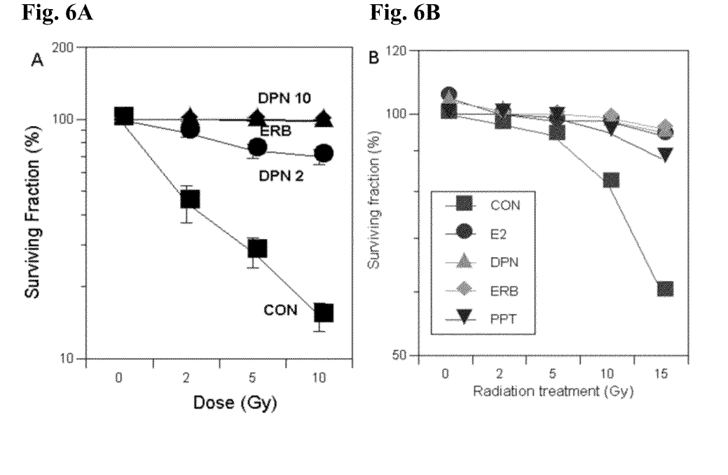

Results show that pre-treatment with ERβ-selective agonists DPN and ERB-041 promotes a dose-dependent increase in lung cell survival post-radiation, while treatment with DPN and ERB-041 at 24 hours after irradiation mitigates these effects in HUVEC cells (FIGS. 6A-6B; P<0.001). In two experiments, treatment with DPN at 24 hrs after irradiation of H23 cells elicited a comparable effect as a radiation mitigator as compared to controls (P<0.001).

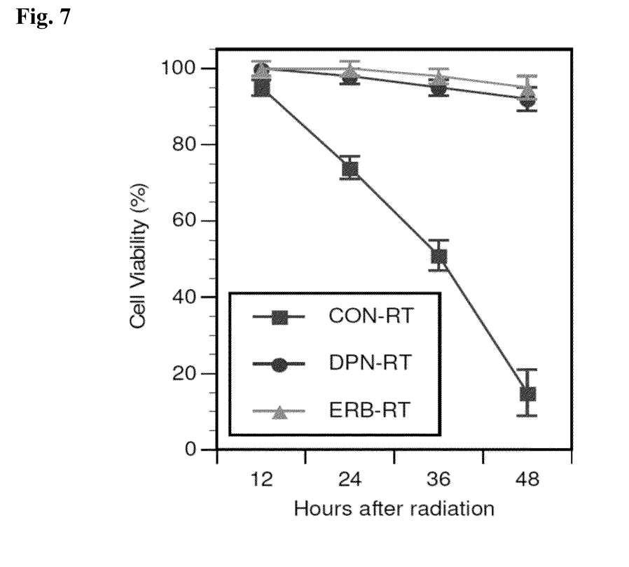

An additional experiment was done using normal human bronchial epithelial (NHBE) cells (see FIG. 7). In this study, DPN and ERB-041 were both found to similarly protect normal epithelial cells from radiation injury. These collective findings support the underlying hypothesis on the role of ERβ signaling in modulating radiation sensitivity.

### 4. SUPPRESSION OF ERβ EXPRESSION IN EPITHELIAL CELLS REDUCES THE RADIOPROTECTIVE/RADIATION MITIGATOR EFFECTS OF ESTROGENS

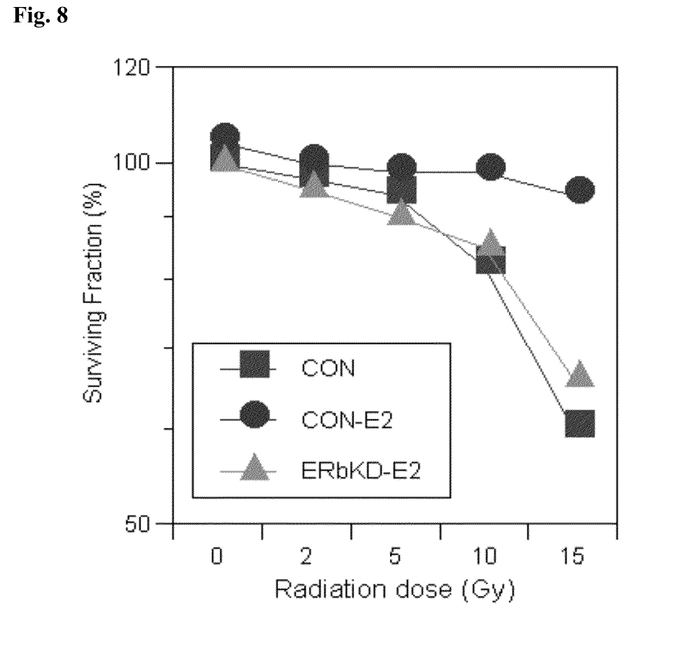

To further assess the role of ERβ as a radioprotector and potential radiation mitigator, lung epithelial cells were treated with small inhibitory RNAs (siRNA) to suppress ERβ expression using established methods (Márquez-Garbán D C, Chen H W, Fishbein M C, Goodglick L, Pietras R J. Steroids. 2007 February; 72(2):135-43. Epub 2007 Feb. 5; Márquez D C, Chen H W, Curran E M, Welshons W V, Pietras R J. Mol Cell Endocrinol. 2006 Feb. 26; 246(1-2):91-100. Epub 2006 Jan. 4). Compared to cells with normal levels of ERβ expression, cells with ERβ knockdowns failed to show a radioprotective effect with estrogen therapy (FIG. 8). Thus, ERβ appears to play a critical role in mediating effects of estrogen post-irradiation.

### 5. ERβ-LIGANDS PROMOTE DNA REPAIR POST-RADIATION

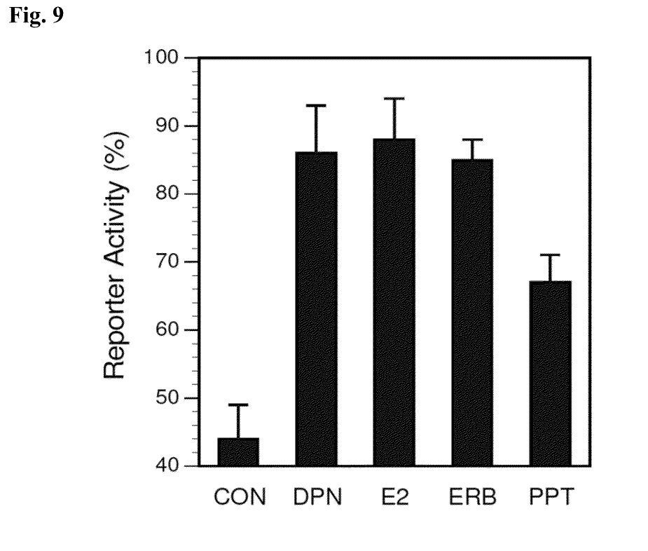

To evaluate a mechanism for radioprotective and potential radiation mitigator effects of E2, DPN and ERB ligands, DNA repair assays were performed using lung H23 cells and an established reporter DNA strategy (Pietras R J, Poen J C, Gallardo D, Wongvipat P N, Lee H J, Slamon D J. Cancer Res. 1999 Mar. 15; 59(6):1347-55). Initial data in FIG. 9 suggest that radioprotective/mitigator activity of ERβ ligands and E2 may be due, in part, to stimulation of enhanced DNA repair after irradiation.

### 6. ESTROGENS MODULATE RT-INDUCED ATM ACTIVATION

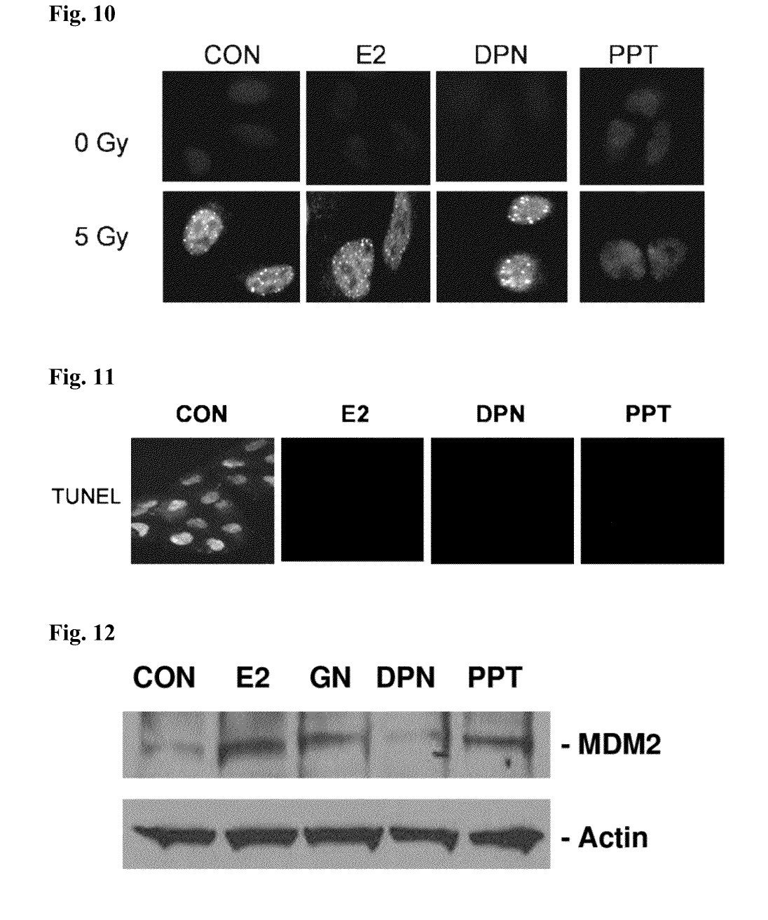

Phosphoryaltion of ATM is a critical first step in cell responses to RT (FIG. 3). Lung cells treated with control or estrogen for 2 hr were then irradiated and examined after 2 h for ATM activation (FIG. 10).

### 7. ESTROGENS REDUCE RADIATION-INDUCED APOPTOSIS

Cell death is a measurable outcome due to significant radiation injury. The effects of estrogens on apoptosis after irradiation were assessed and estradiol, DPN and PPT showed efficacy in suppressing radiation-induced apoptosis (FIG. 11).

### 8. ESTROGENS MODULATE MDM2 EXPRESSION IN LUNG CELLS

Estrogens modulate MDM2 expression in lung cells. MDM2 is well known to bind and inhibit p53 FIG. 3). ERα binds and regulates the MDM2 promoter to stimulate MDM2 expression (see FIG. 12). Indeed, MDM2 SNP309 is reported to occur in an ER binding region of the MDM2 promoter, and affected individuals with SNP309 have increased MDM2 levels mediated by estrogen signaling (33,34). It is notable that that E2 and PPT, but not DPN, stimulate MDM2 levels in lung cells (FIG. 12). This result suggests that this function of estrogen is likely controlled more by ERα than by ERβ signaling in the lung epithelial cells. Hence, selection of effects specific for ERβ may be advantageous for radiation mitigation.

### 9. ESTROGENS STIMULATE VEGF SECRETION BY IRRADIATED LUNG EPITHELIAL CELLS TO POTENTIALLY IMPACT NEIGHBORING VASCULAR TISSUES

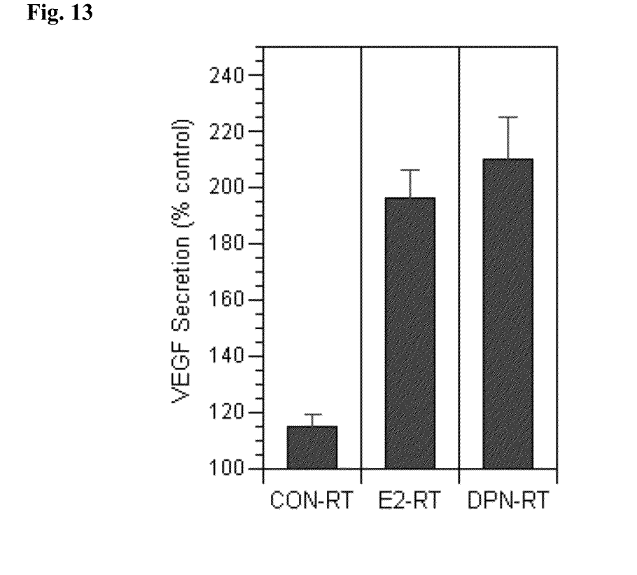

Following irradiation, lung epithelial cells pre-treated with either E2 or DPN show a 2-fold increase in vascular endothelial growth factor (VEGF) secretion (P<0.001; see FIG. 13). Vascular endothelial growth factor may then promote systemic effects of E2/DPN ligands in support of tissue viability. In three experiments recently completed, it was found that treatment with DPN at 24 hours after irradiation (10 Gy) elicits a comparable increase in VEGF secretion as assayed by ELISA methods as compared with controls (P<0.001).

### 10. ERβ AGONISTS ARE RADIATION MITIGATORS IN ANIMAL MODELS

C3H mice were bred and maintained in a strict defined-flora, pathogen-free environment in the AALAC-accredited animal facilities. The UCLA ARC approved all experiments which were done in accord with all local and national guidelines for care and use of animals. Male mice, 9-10 wks old, received 7.725 Gy TBI from a Gamma Cell 40 irradiator (¹³⁷Cs source; Atomic Energy of Canada) at a dose rate of 67 cGy/min. For mitigation, drug or vehicle was given subcutaneously (diarylpropionitrile [DPN], 10 mg/kg) or by oral gavage (prinaberel [ERB-041], 50 mg/kg) daily for five days starting 24 h after TBI. Mice were monitored for up to 60 days using standard criteria for humane euthanasia as an endpoint and weights were recorded twice/week. DPN protects mice from lethality and improves survival (P<0.001), while orally administered ERB-041 is somewhat less efficacious as a radiation mitigator (P<0.05).

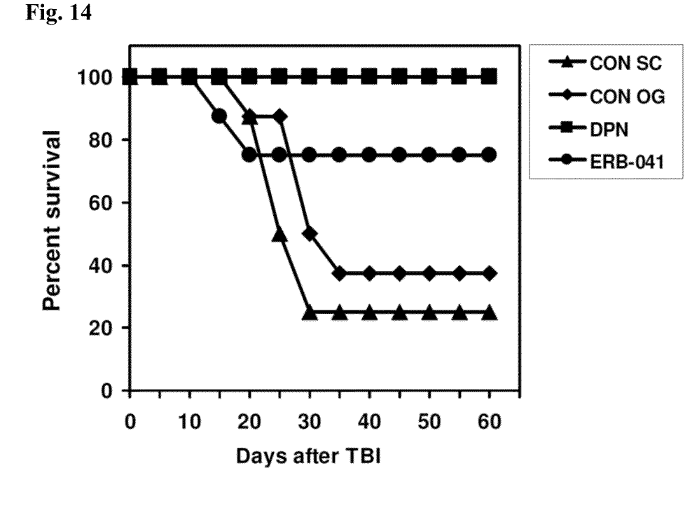

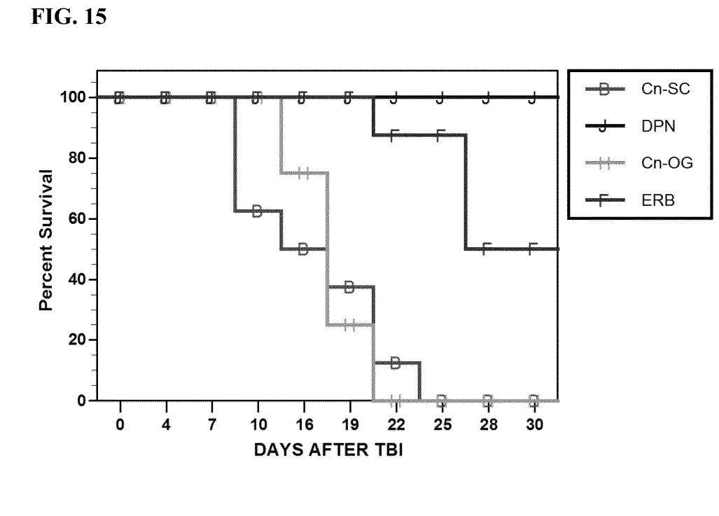

To validate our results above using a different animal model, we used C57/BL6 mice that were also bred and maintained in our AALAC-accredited animal facilities. As above, the UCLA ARC approved all studies. Male mice, 9 weeks-old, in a well-ventilated Lucite chamber without anesthesia received 8.5 Gy TBI from a Gamma Cell 40 irradiator (¹³⁷Cs source; Atomic Energy of Canada) at a dose rate of 67 cGy/min. For mitigation, drug or vehicle was given subcutaneously (diarylpropionitrile [DPN], 10 mg/kg) or by oral gavage (prinaberel [ERB-041], 50 mg/kg) daily for five days starting 24 hours after TBI. Mice were monitored for 30 days using standard criteria for humane euthanasia as an endpoint and weights were recorded twice/week. DPN protects mice from lethality and improves survival (P<0.001), while orally administered ERB-041 is somewhat less efficacious as a radiation mitigator (P<0.05) (FIG. 15). These preliminary findings (FIGS. 14 & 15) provide good evidence to pursue further investigation of ERβ ligands as radiation mitigators.

### 11. IDENTIFICATION OF ESTROGEN RECEPTOR (ER)-SELECTIVE LIGANDS THAT PROMOTE RECOVERY AND SURVIVAL OF CRITICAL TISSUES POST-RADIATION AND THE PATHWAYS INVOLVED

Levels of ER transcripts and protein are assayed in vitro after irradiation and in cells treated with or without ERβ-ligands (Pietras R J, Poen J C, Gallardo D, Wongvipat P N, Lee H J, Slamon D J. Cancer Res. 1999 Mar. 15; 59(6):1347-55; Pietras R J, Márquez D C, Chen H W, Tsai E, Weinberg O, Fishbein M. Steroids. 2005 May-June; 70(5-7):372-81. Epub 2005 Mar. 25; Marquez D C, Lee J, Lin T, Pietras R J. Endocrine. 2001 November; 16(2):73-81; Pietras R J. Breast J. 2003 September-October; 9(5):361-73; Márquez-Garbán D C, Chen H W, Fishbein M C, Goodglick L, Pietras R J. Steroids. 2007 February; 72(2):135-43. Epub 2007 Feb. 5). Cells are treated with 0, 2, 4, 6, 8 Gy X-rays at a dose rate of 1 Gy/min and cultured for 5 days and ER expression assayed everyday for 5 days post exposure. Prior work suggests that ERβ is selectively upregulated by irradiation and may be a protective tissue response to RT (Zhou Y, Mi M T (2005). J Radiat Res. 46(4):425-33).

For ERβ-ligand studies control vehicle and ligands selective for activating ERβ are used as follows (FIG. 5; Harrington W R, Sheng S, Barnett D H, Petz L N, Katzenellenbogen J A, Katzenellenbogen B S. Mol Cell Endocrinol. 2003 Aug. 29; 206(1-2):13-22; Mishra R G, Stanczyk F Z, Burry K A, Oparil S, Katzenellenbogen B S, Nealen M L, Katzenellenbogen J A, Hermsmeyer R K. Am J Physiol Heart Circ Physiol. 2006 January; 290(1):H295-303. Epub 2005 Sep. 30; Patisaul H B, Melby M, Whitten P L, Young L J. Endocrinology. 2002 June; 143(6):2189-97; Weihua Z, Makela S, Andersson L C, Salmi S, Saji S, Webster J I, Jensen E V, Nilsson S, Warner M, Gustafsson J A. Proc Natl Acad Sci USA. 2001 May 22; 98(11):6330-5): DPN and ERB-041 (see FIG. 5). Control ligands include: PPT (FIG. 5) (ERα agonist) and PHTPP (ERβ antagonist). ERα and ERβ are assessed by immunoassay and immunofluorescence (Pietras R J, Arboleda J, Reese D M, Wongvipat N, Pegram M D, Ramos L, Gorman C M, Parker M G, Sliwkowski M X, Slamon D J. Oncogene. 1995 Jun. 15; 10(12):2435-46; Márquez-Garbán D C, Chen H W, Fishbein M C, Goodglick L, Pietras R J. Steroids. 2007 February; 72(2):135-43. Epub 2007 Feb. 5; Márquez D C, Chen H W, Curran E M, Welshons W V, Pietras R J. Mol Cell Endocrinol. 2006 Feb. 26; 246(1-2):91-100. Epub 2006 Jan. 4). ERβ antibodies include 14C8 (Abcam), ERβ1, ERβ2 (Serotec); ERα antibodies are 1D5 (Zymed) (Lau S K, Chu P G, Weiss L M. Appl Immunohistochem Mol Morphol. 2006 March; 14(1):83-7). ER-null cells or cells with ER knockdowns (Márquez-Garbán D C, Chen H W, Fishbein M C, Goodglick L, Pietras R J. Steroids. 2007 February; 72(2):135-43. Epub 2007 Feb. 5) are used as controls. Ligands are added starting 24 hrs after irradiation. These studies reveal levels/types of ER forms that occur post-irradiation and post ligand binding.

Human lung cells, such as H23, A549 (ATCC) (19,31,39), are routinely cultured in RPMI 1640 media with 10% FBS (Márquez-Garbán D C, Chen H W, Fishbein M C, Goodglick L, Pietras R J. Steroids. 2007 February; 72(2):135-43. Epub 2007 Feb. 5). NHBE (FIG. 7) and HUVEC are cultured by manufacturer's recommendations (Li D, Williams J I, Pietras R J (2002). Oncogene. 21(18):2805-14). Additional lung (murine and human) and colon cell lines are being screened (e.g. HCC-2998 [Xu X, Veenstra T D. Genome Med. 2012 Apr. 30; 4(4):311; and those available from commercial suppliers) for ERβ expression levels and ligand interactions. For E2-free conditions, media are changed 48 h before studies to phenol-red free media with 1% dextran-coated, charcoal-treated (DCC)—FBS (Song R X, Barnes C J, Zhang Z, Bao Y, Kumar R, Santen R J. Proc Natl Acad Sci USA. 2004 Feb. 17; 101(7):2076-81. Epub 2004 Feb. 5; Marquez D C, Chen H W, Curran E M, Welshons W V, Pietras R J. Mol Cell Endocrinol. 2006 Feb. 26; 246(1-2):91-100. Epub 2006 Jan. 4). Panels of cells including NHBE, HUVEC and A549 cells can be used for screening assays. Additional cell lines (e.g., HMEC, HInEC) can be incorporated to confirm and extend initial studies.

### 12. APOPTOSIS MEASUREMENTS BY TUNEL ASSAY

Tests for apoptosis can be conducted by TUNEL assay (Li D, Williams J I, Pietras R J (2002). Oncogene. 21(18):2805-14). Although radiation is generally believed not a good apoptotic stimuli in other than hematopoietic cells, this may change in the context of ER signaling. To test this, 0, 2, 5 and 10 Gy-irradiated cells are treated, then 24 h later treated with selected agents (DPN, ERB-041) followed by TUNEL assays every day for 4 days post-irradiation. A confirmatory assay can be the measure of 85-kD cleaved PARP protein, another marker to detect apoptotic cells, by Western blot of nuclear extracts (Raffoul J J, Wang Y, Kucuk O, Forman J D, Sarkar F H, Hillman G G (2006). BMC Cancer. 6:107). To assess apoptosis mechanisms, early changes in AKT are assayed (anti-apoptosis mediator; Márquez-Garbán D C, Chen H W, Fishbein M C, Goodglick L, Pietras R J. Steroids. 2007 February; 72(2):135-43. Epub 2007 Feb. 5; Marquez D C, Chen H W, Curran E M, Welshons W V, Pietras R J. Mol Cell Endocrinol. 2006 Feb. 26; 246(1-2):91-100. Epub 2006 Jan. 4) along with mitochondrial elements (Bax, Bcl-2, activated caspase-3) in treated and control cells (Nübel T, Damrot J, Roos W P, Kaina B, Fritz G. Clin Cancer Res. 2006 Feb. 1; 12(3 Pt 1):933-9). Although DPN appears to reduce radiation-induced apoptosis in lung cells, it is important to determine apoptosis effects in other cell types as this effect may vary in other cells such as colon depending on tissue-specific ERβ levels (Torlakovic E, Lilleby W, Berner A, Torlakovic G, Chibbar R, Furre T, Fosså S D (2005). Int J Cancer. 117(3):381-6).

### 13. CLONOGENICITY ASSAY

Clonogenicity (Brush J, Lipnick S L, Phillips T, Sitko J, McDonald J T, McBride W H. Semin Radiat Oncol. 2007 April; 17(2):121-30; Sarkaria J N, Tibbetts R S, Busby E C, Kennedy A P, Hill D E, Abraham R T. Cancer Res. 1998 Oct. 1; 58(19):4375-82) and cell proliferation (Li D, Williams J I, Pietras R J (2002). Oncogene. 21(18):2805-14; Márquez-Garbán D C, Chen H W, Fishbein M C, Goodglick L, Pietras R J. Steroids. 2007 February; 72(2):135-43. Epub 2007 Feb. 5). The most robust assay to determine the effect of ERβ ligands on radiation responses in vitro is the standard clonogenic assay, which can be performed on the cell line panels. Cells are grown in the exponential phase, irradiated, and re-plated with or without the ERβ ligands described herein. Surviving cells can be assessed by the fact that they maintain their capacity to form colonies of >50 cells as visualized by staining with crystal violet. Since irradiation-induced activation of EGFR increases proliferation by activating MAPK pathways (Sturla L M, Amorino G, Alexander M S, Mikkelsen R B, Valerie K, Schmidt-Ullrichr R K. J Biol Chem. 2005 Apr. 15; 280(15):14597-604. Epub 2005 February; Das A K, Chen B P, Story M D, Sato M, Minna J D, Chen D J, Nirodi C S. Cancer Res. 2007 Jun. 1; 67(11):5267-74), early MAPK activation can be assessed (Márquez-Garbán D C, Chen H W, Fishbein M C, Goodglick L, Pietras R J. Steroids. 2007 February; 72(2):135-43. Epub 2007 Feb. 5; Márquez D C, Chen H W, Curran E M, Welshons W V, Pietras R J. Mol Cell Endocrinol. 2006 Feb. 26; 246(1-2):91-100. Epub 2006 Jan. 4) after irradiation of cells treated with and without ERβ ligands given 24, 48 and 72 hrs after irradiation. The ATPlite assay is also used to determine the extent of cell proliferation directly. It is expected that the ERβ agonist can increase proliferation and rescue clonogenicity after irradiation.

### 14. MODULATION OF POST-RT CELL CYCLE PROGRESSION AND DNA REPAIR

Modulation of post-RT cell cycle progression (Sarkaria J N, Tibbetts R S, Busby E C, Kennedy A P, Hill D E, Abraham R T. Cancer Res. 1998 Oct. 1; 58(19):4375-82) and DNA repair (Pietras R J, Poen J C, Gallardo D, Wongvipat P N, Lee H J, Slamon D J. Cancer Res. 1999 Mar. 15; 59(6):1347-55; Das A K, Chen B P, Story M D, Sato M, Minna J D, Chen D J, Nirodi C S. Cancer Res. 2007 Jun. 1; 67(11):5267-74) by E2 and ERβ ligands given 24 h post-RT can be done in irradiated cells as before (FIG. 9). This can be confirmed by gH2AX staining and FACS analysis. The regulation of p53 and p21/WAF1 signaling and expression of specific DNA repair enzymes can be pursued (Pietras R J, Poen J C, Gallardo D, Wongvipat P N, Lee H J, Slamon D J. Cancer Res. 1999 Mar. 15; 59(6):1347-55; Das A K, Chen B P, Story M D, Sato M, Minna J D, Chen D J, Nirodi C S. Cancer Res. 2007 Jun. 1; 67(11):5267-74; Sarkaria J N, Tibbetts R S, Busby E C, Kennedy A P, Hill D E, Abraham R T. Cancer Res. 1998 Oct. 1; 58(19):4375-82) to complete the picture of how ERβ modulates DNA repair (Pietras R J, Poen J C, Gallardo D, Wongvipat P N, Lee H J, Slamon D J. Cancer Res. 1999 Mar. 15; 59(6):1347-55).

### 15. INCREASED GROWTH FACTOR PRODUCTION/SECRETION

Increased growth factor production/secretion (23,24). E2 stimulates secretion of several EGFR family ligands (Pietras R J, Marquez-Garban D C. (2007). Clin Cancer Res 13:4672-6; Márquez-Garbán D C, Chen H W, Fishbein M C, Goodglick L, Pietras R J. Steroids. 2007 February; 72(2):135-43. Epub 2007 Feb. 5; Marquez D C, Chen H W, Curran E M, Welshons W V, Pietras R J. Mol Cell Endocrinol. 2006 Feb. 26; 246(1-2):91-100. Epub 2006 Jan. 4; Aguilar Z, Akita R W, Finn R S, Ramos B L, Pegram M D, Kabbinavar F F, Pietras R J, Pisacane P, Sliwkowski M X, Slamon D J. Oncogene. 1999 Oct. 28; 18(44):6050-62) and VEGF by lung cells (FIG. 13; Li D, Williams J I, Pietras R J (2002). Oncogene. 21(18):2805-14; Petit A M, Rak J, Hung M C, Rockwell P, Goldstein N, Fendly B, Kerbel R S. Am J Pathol. 1997 December; 151(6):1523-30). These E2-induced ligands may activate critical downstream receptors to regulate response to RT. ELISA methods can be used to find effects of ERβ-ligands (24 h post-RT) on production of EGFR/VEGFR ligands in cells with/without irradiation (Li D, Williams J I, Pietras R J (2002). Oncogene. 21(18):2805-14; Petit A M, Rak J, Hung M C, Rockwell P, Goldstein N, Fendly B, Kerbel R S. Am J Pathol. 1997 December; 151(6):1523-30; Aguilar Z, Akita R W, Finn R S, Ramos B L, Pegram M D, Kabbinavar F F, Pietras R J, Pisacane P, Sliwkowski M X, Slamon D J. Oncogene. 1999 Oct. 28; 18(44):6050-62).

### 16. DECIPHER CELL ER SIGNALING MODES AFTER RT USING ER siRNA

Decipher cell ER signaling modes after RT using ER siRNA's to selectively suppress ERα or ERβ. siRNA expression vectors can be used to downregulate ER expression (FIG. 8). Treatment of lung cells with siRNA directed to ERα and ERβ effectively reduces mRNA expression of ERα and ERβ, respectively, but control protein levels do not change (Marquez-Garbán D C, Chen H W, Fishbein M C, Goodglick L, Pietras R J. Steroids. 2007 February; 72(2):135-43. Epub 2007 Feb. 5; Marquez D C, Chen H W, Curran E M, Welshons W V, Pietras R J. Mol Cell Endocrinol. 2006 Feb. 26; 246(1-2):91-100. Epub 2006 Jan. 4). Further, nonspecific siRNAs exert no effect. Radiation mitigation actions of E2 and ERβ agonists can be tested (with methods as in FIG. 8) using this knockdown system to confirm the hypothesis that ERβ is an obligate pathway to mediate cell survival after irradiation.

### 17. ER-SELECTIVE LIGANDS AS RADIATION MITIGATORS

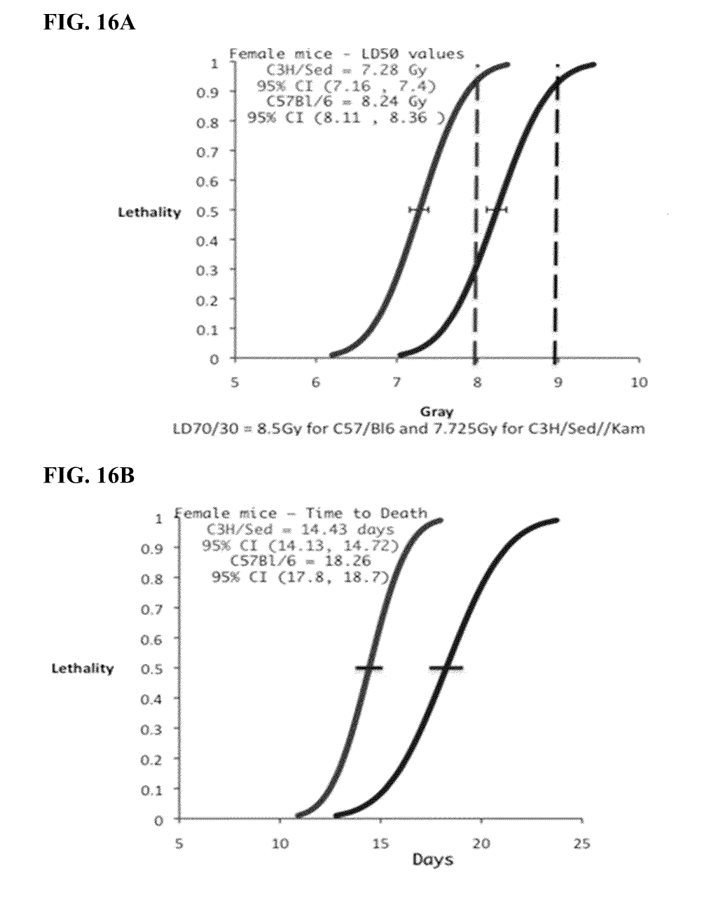

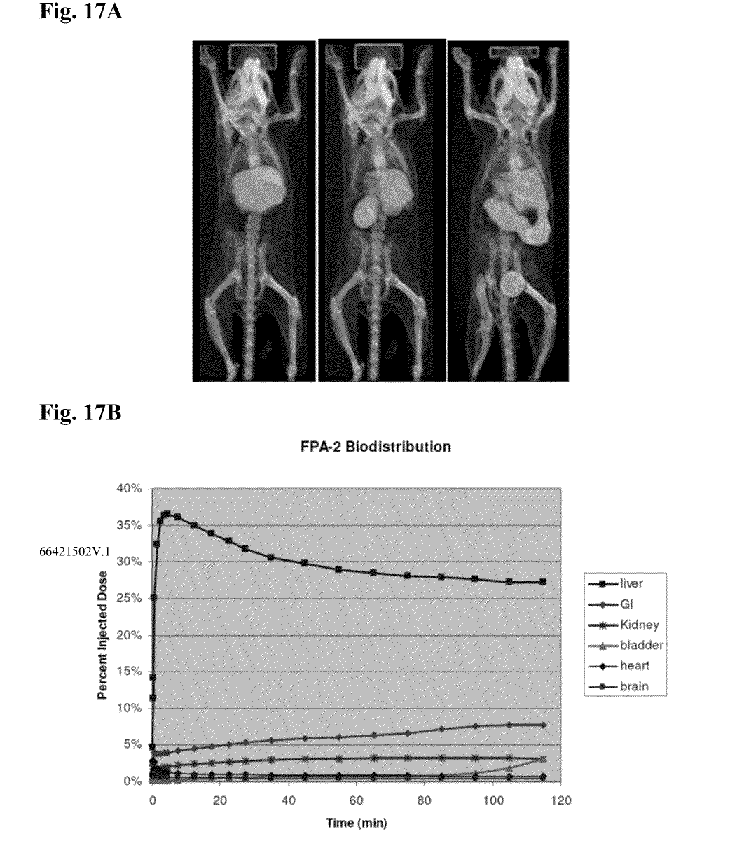

One can develop ER-selective ligands as radiation mitigators administered after radiation exposure, with an emphasis on broad activity to reduce major morbidity and promote survival using animal models. Promising radiation mitigators detected in vitro can be used for in vivo testing in mice exposed to LD70/30 lethal irradiation. See e.g., FIGS. 16A-16B. Based on preliminary findings, ERβ1 agonist DPN is a primary focus of these studies which include: a) studies of candidate drug (e.g. DPN, ERB-041) doses, routes of administration and schedules at 24, 48 and 72 hours post-irradiation can be performed in vivo using C3H and C57BI/6 survival models, whose response to radiation is known (see FIG. 15); b) alternate derivatives of DPN to enhance activity as a radiation mitigator with increased bioavailability; and c) pharmacokinetic studies using established methods known in the art. Studies of drug biodistribution can be done in vivo using small animal imaging with PET. See e.g., FIG. 17A-17B.

Survival experiments using animal models to further assess ERβ-selective ligands as radiation mitigators. Male 10-week old C3H/Sed//Kam and C57Bl/6 mice are bred and maintained in a defined-flora, pathogen-free environment in the American Association of Laboratory Animal Care (AALAC)— accredited Animal Facility of the UCLA Department of Radiation Oncology (Kim K, Damoiseaux R, Norris A J, Rivina L, Bradley K, Jung M E, Gatti R A, Schiestl R H, McBride W H. Int J Radiat Biol. 2011 August; 87(8):839-45. Epub 2011 Mar. 14.; Kim K, Pollard J M, Norris A J, McDonald J T, Sun Y, Micewicz E, Pettijohn K, Damoiseaux R, Iwamoto K S, Sayre J W, Price B D, Gatti R A, McBride W H. Clin Cancer Res. 2009 Dec. 1; 15(23):7238-45.). Initially C3H mice can be used and effective agents re-tested in the C57Bl/6 strain to establish if there is any genetic dependency. For LD70/30 experiments, C3H mice are positioned in a Lucite jig and irradiated with 7.725Gy using a Gamma cell 40 irradiator (Cs-137 source; Atomic Energy of Canada Ltd., Ottawa, Canada) at a dose rate of approximately 67cGy/min. Irradiated mice can then be randomized to receive vehicle, DPN or ERB-041 at varying doses by subcutaneous injection (s.c.) or oral gavage respectively, beginning 24 h after TBI and continued every day for 5 days (8 mice/group). In the basic proposed treatment protocol, mice can be injected s.c. daily with ERβ ligand DPN [2,3-bis(4-hydroxyphenyl)-propionitrile] at 5-10 mg/kg/mouse (Mishra R G, Stanczyk F Z, Burry K A, Oparil S, Katzenellenbogen B S, Nealen M L, Katzenellenbogen J A, Hermsmeyer R K. Am J Physiol Heart Circ Physiol. 2006 January; 290(1):H295-303. Epub 2005 Sep. 30) or vehicle control. Similarly, the ERβ ligand ERB-041 can be administered by oral gavage at 50-75 mg/kg/mouse daily using the same treatment schedule. Study animals can be monitored for general health and weight loss every 2 days for 30-40 days or until they are moribund. During critical times (12-20 days after radiation) animals can be monitored twice daily for signs of morbidity. Kaplan-Meier survival curves, median survival, and lethality at 30 days can be recorded (Maclachlan T, Narayanan B, Gerlach V L, Smithson G, Gerwien R W, Folkerts O, Fey E G, Watkins B, Seed T, Alvarez E. Int J Radiat Biol. 2005 August; 81(8):567-79; Kodell R L, Lensing S Y, Landes R D, Kumar K S, Hauer-Jensen M (2010). Biometrics 66:239-248. PMCID: PMC3036987). In all studies, samples of lung, vascular, bone marrow and intestinal tissues of mice can be collected postmortem using established methods (Zhou Y, Mi M T (2005). J Radiat Res. 46(4):425-33; Landauer M R, Srinivasan V, Seed T M (2003). J Appl Toxicol. 23(6):379-85; Davis T A, Clarke T K, Mog S R, Landauer M R (2007). Int J Radiat Biol. 83(3):141-51; Kim K, Damoiseaux R, Norris A J, Rivina L, Bradley K, Jung M E, Gatti R A, Schiestl R H, McBride W H. Int J Radiat Biol. 2011 August; 87(8):839-45. Epub 2011 Mar. 14) after animals are euthanized at the termination of the experiment for further analyses. Agents that increase survival to >90% by these schedules can be tested to establish their optimal dose and schedule delivery and for their dose modifying factor (DMF). Single doses can be compared with multiple doses and s.c. with p.o. routes. High single doses can be used to establish toxicity. To establish the DMF, C3H mice can be exposed to graded total-body radiation doses over a range of doses from 7.725-9.5Gy in 0.5Gy increments.

General Design of in vivo Animal Studies. Planned groups for the several treatments include mice treated with control vehicle (CON) or ERβ ligands 1 (ERB1=DPN) or 2 (ERB2=ERB-041):

TBI at 3 different doses of ERB1 or ERB2 including the following groups with 8 mice per group: CON, ERB1 (dose 1), ERB1 (dose 2), ERB1 (dose 3), ERB2 (dose 1), ERB2 (dose 2), ERB2 (dose 3), with a total of 56 mice for these experiments. Different routes of drug administration (s.c. versus p.o.) after TBI may also be determined depending on preliminary data.

Once optimal drug doses are determined at 24 hours post-TBI, the most promising drug candidate at its optimal dose can be used to assess additional post-TBI time windows by giving drug at 24, 48, 72 h after TBI (using 8 mice/group). Time of ERB administration for 5 days after TBI variable, with 8 mice per group: CON, ERB 24 h post, ERB 48 h post, ERB 72 h post, with a total of 32 mice for these experiments.

Efficacy of ERB dosing for 1, 3 or 5 days after TBI, with 8 mice per group: CON, ERB 1 day, ERB 3 days, ERB 5 days, with a total of 32 mice for these experiments.

TBI at different doses with optimal ERB radiation mitigator, with 8 mice per group: CON, TBI 1, TBI 2, TBI 3, with a total of 32 mice for these experiments.

Experiments above performed using C3H can also be completed on male C57BI/6 mice whole-body irradiated with tentatively 8.5 Gy. Study animals can be monitored for up to 30-40 days after TBI, and the number of moribund/dead mice can be recorded. Kaplan-Meier survival curves, median survival times and lethality at 30-40 days can be recorded. Radiobiology/postmortem tissue assays can also be undertaken (Zhou Y, Mi M T (2005). J Radiat Res. 46(4):425-33; Landauer M R, Srinivasan V, Seed T M (2003). J Appl Toxicol. 23(6):379-85; Davis T A, Clarke T K, Mog S R, Landauer M R (2007). Int J Radiat Biol. 83(3):141-51).

8 mice/group can be used in preliminary studies to screen dosages and schedules (Brush J, Lipnick S L, Phillips T, Sitko J, McDonald J T, McBride W H. Semin Radiat Oncol. 2007 April; 17(2):121-30; Kim K, Damoiseaux R, Norris A J, Rivina L, Bradley K, Jung M E, Gatti R A, Schiestl R H, McBride W H. Int J Radiat Biol. 2011 August; 87(8):839-45. Epub 2011 Mar. 14.; Suman S, Maniar M, Fornace A J Jr, Datta K. Radiat Oncol. 2012 Jan. 20; 7:6.). However, for definitive studies with the final chosen agent, the sample size can be 24 mice in each group. Using an LD70/30 model, this is based on the assumption that controls can have 30% survival rate and treatment conditions can have 90% survival rate. Based on these assumptions a 0.050 level two-sided log-rank test for equality of survival curves can have 95% power to detect the difference between the groups. Mice are commonly used to assess effects of irradiation (Brush J, Lipnick S L, Phillips T, Sitko J, McDonald J T, McBride W H. Semin Radiat Oncol. 2007 April; 17(2):121-30; Epperly M, Jin S, Nie S, Cao S, Zhang X, Franicola D, Wang H, Fink M P, Greenberger J S. Radiat Res. 2007 November; 168(5):552-9). It is predicted that about 300 mice are needed, with 150 C3H and 150 C57Bl/6 mice, in the first year of the study and similar numbers in succeeding years. At this time, DPN is the selected candidate as a radiation mitigator to be effectively given at least 24 hr after TBI.

The mice (8-10 weeks of age) can be male C3H and C57BI/6 mice. Mice can be treated with total body irradiation (TBI) with and without post-TBI administration of radiation mitigators. TBI can range from 7.725 to 8.5 Gy from a Gamma Cell 40 irradiator (137Cs source; Atomic Energy of Canada) at established dose rates. Radiation mitigators can be administered at varying doses and schedules to define optimal treatment conditions. Initially, radiation mitigators can be given daily for five days, starting at 24 h after TBI. Mice can be monitored for 30-40 days using standard criteria for humane euthanasia as an endpoint. These studies can also include a limited set of studies to assess blood and tissue changes as a consequence of radiation mitigator treatment post-TBI. Studies of the radiation mitigation effects of selected estrogen receptor-beta ligands (DPN, ERB-041) can be tested in mice. Groups for the several treatments in irradiated mice can include the following:

C3H mice treated with control vehicle (CON) or ERβ. ligands 1 (ERB1) or 2 (ERB2):

A. TBI at 3 different doses of ERB1 or ERB2: Mice TBI at 3 different doses of ERB1 or ERB2:

| | |
|---|---|
| CON | 8 mice |
| ERB1 (dose 1) | 8 mice |
| ERB1 (dose 2) | 8 mice |
| ERB1 (dose 3) | 8 mice |
| ERB2 (dose 1) | 8 mice |
| ERB2 (dose 2) | 8 mice |
| ERB2 (dose 3) | 8 mice |
| Total | 56 mice |

B. Once optimal drug doses are determined at 24 hours post-TBI, the most promising drug candidate at its optimal dose can be used to assess additional post-TBI time windows by giving drug at 24, 48, 72 h after TBI:

| | |
|---|---|
| CON | 8 mice |
| ERB- 24 h post | 8 mice |
| ERB- 48 h post | 8 mice |
| ERB- 72 h post | 8 mice |
| Total | 32 mice |

C. Efficacy of ERB dosing for 1, 3 and 5 days after TBI:

| | |
|---|---|
| CON | 8 mice |
| ERB 1 day | 8 mice |
| ERB 3 days | 8 mice |
| ERB 5 days | 8 mice |
| Total | 32 mice |

D. TBI at different doses with optimal ERB radiation mitigator (ERB) with 8 mice/group:

| | |
|---|---|
| CON | 8 mice |
| TBI1 | 8 mice |
| TBI2 | 8 mice |
| TBI3 | 8 mice |
| Total | 32 mice |

After the studies above are concluded successfully, experiments can be repeated using C57BI/6 mice treated with control vehicle (CON) or ERβ. ligands 1 (ERB1) or 2 (ERB2) to confirm that the findings observed are not strain-dependent.

In the proposed treatment protocol above mice can be injected subcutaneously (s.c) daily with ER-beta ligand (e.g. 2,3-bis(4-hydroxyphenyl)-propionitrile at 5-10 mg/kg/mouse [Mishra R et al. Am J. Physiol. Heart Circ. Physiol. 290: H295-303, 2006]) for 5 days or vehicle control, following total body irradiation (TBI) using established doses and modes as reported previously (see Epperly M et al. Radiat Res 168: 552, 2007; Liu W-C et al. Radiat Res 166:900, 2006; Zhou Y, Mi M-T. J Radiat Res 46: 425, 2005). Similarly, the ER-beta ligand ERB can be administered by oral gavage at 50-75 mg/kg/mouse daily using the same treatment schedule. The number of surviving mice can be recorded daily for 30 days after TBI. Mortality can be recorded in each group, with survival rates calculated on a 30-day post-exposure period. In all studies, samples of selected mouse tissues can be collected postmortem after animals are euthanized with isoflurane at the termination of the experiment for potential future analyses (Kim K et al. Int J Radiat Biol. 87:839, 2011). As noted above, mice are randomized to different drug treatment groups with controls for up to 30 days after total body irradiation.

### 18. SAMPLING OF SYSTEMIC BLOOD AND MOUSE TISSUES

Sampling of Systemic Blood and Mouse Tissues: C3H mice can be exposed to tentatively 8.5 Gy TBI and receive treatment 24 or 48 hrs post-TBI with vehicle or DPN (or best candidate drug) s.c. for 2-5 doses. Terminal anesthesia can be used to collect tissue and blood samples from mice. Mitigation of hematopoietic toxicity by DPN can be investigated by peripheral white blood cell and platelet counts at 0, 3, 7, 21 and 28 d after radiation, with blood samples (5 mice/group/time point) collected by cardiac puncture in EDTA tubes and subjected to complete blood counts (Suman S, Maniar M, Fornace A J Jr, Datta K. Radiat Oncol. 2012; 7:6). For granulocyte-macrophage colony forming unit (GM-CFU) assays, mice (5/group) can be euthanized at 7d after TBI. Femurs can be excised and bone marrow cells isolated (Suman S et al. Radiat Oncol. 2012; 7:6). Other organs such as lungs and intestines, liver and spleen can be perfused with PBS and extracted postmortem.

Sampling of Systemic Blood and Mouse Tissues (Brown S L, Kolozsvary A, Liu J, Ryu S, Kim J H. Radiat Res. 2008 April; 169(4):474-8); Chen B J, Deoliveira D, Spasojevic I Sempowski G D, Jiang C, Owzar K, Wang X, Gesty-Palmer D, Cline J M, Bourland J D, Dugan G, Meadows S K, Daher P, Muramoto G, Chute J P, Chao N J. PLoS One 2010; 5:e11056; Zhou D, Deoliveira D, Kang Y, Choi S S, Li Z, Chao N J, Chen B J. In J Radiat Oncol Biol Bhys. 2012, Sep. 25, [Epub ahead of print]; Suman S, Maniar M, Fornace A J Jr, Datta K. Radiat Oncol. 2012 Jan. 20; 7:6.). C3H mice can be exposed to 8.5 Gy TBI and receive treatment 24 or 48 hr later with vehicle or DPN s.c. for 2-5 doses. Terminal anesthesia can be used to collect tissue and blood samples from mice. Mitigation of hematopoietic toxicity by DPN can be investigated by peripheral white blood cell and platelet counts at 0, 3, 7, 21 and 28 d after radiation, with blood samples (5 mice/group/time point) collected by cardiac puncture in EDTA tubes and subjected to complete blood counts (Brown S L, Kolozsvary A, Liu J, Ryu S, Kim J H. Radiat Res. 2008 April; 169(4):474-8); Chen B J, Deoliveira D, Spasojevic I Sempowski G D, Jiang C, Owzar K, Wang X, Gesty-Palmer D, Cline J M, Bourland J D, Dugan G, Meadows S K, Daher P, Muramoto G, Chute J P, Chao N J. PLoS One 2010; 5:e11056; Zhou D, Deoliveira D, Kang Y, Choi S S, Li Z, Chao N J, Chen B J. In J Radiat Oncol Biol Bhys. 2012, Sep. 25, [Epub ahead of print]; Suman S, Maniar M, Fornace A J Jr, Datta K. Radiat Oncol. 2012 Jan. 20; 7:6.). For granulocyte-macrophage colony forming unit (GM-CFU) assays, mice (5/group) can be euthanized at 7d after TBI. Femurs can be excised and bone marrow cells isolated, counted, then plated in triplicate in ultra-low attachment 60-mm dishes (Corning) using methocult (M3534, StemCell Technologies) medium supplemented with 10 ng/mL GM-CSF. Plates can be incubated 37° C. for 7d, and colonies can be counted using a dissecting microscope (Suman S, Maniar M, Fornace A J Jr, Datta K. Radiat Oncol. 2012 Jan. 20; 7:6.). Bone marrow (5 mice/group) can also be used for histopathologic analysis. Surgically-removed femurs from each mouse at 7d after TBI can be fixed in 10% buffered formalin, decalcified, paraffin embedded, and 5-μm thick sections stained with H&E using standard methods (Mah V, Seligson D B, Li A, Márquez D C, Wistuba I I, Elshimali Y, Fishbein M C, Chia D, Pietras R J, Goodglick L. Cancer Res. 2007 Nov. 1; 67(21):10484-90.). Unstained sections can be used for TUNEL assay at 7d post-TBI. Bone marrow cellularity in H&E stained sections can be semiquantitatively scored (5 mice/each group) by counting nucleated cells (Suman S, Maniar M, Fornace A J Jr, Datta K. Radiat Oncol. 2012 Jan. 20; 7:6. PMCID: PMC3275448). The DNA damage response pathway involving ATM and p53 can also be investigated by Western blot in bone marrow cells at 7d post-exposure (Suman S, Maniar M, Fornace A J Jr, Datta K. Radiat Oncol. 2012 Jan. 20; 7:6.). Hematopoietic cells are among the most sensitive to radiation, and mice often die of sequelae of hematopoietic and immune failure. It is expected that treatment with DPN can increase recovery of hematopoietic cells. In these experiments, additional tissue specimens can also be harvested from treatment groups noted above, including intestine/colon and lung, for fixation and paraffin embedding (Subhrajit Saha, Payel Bhanja, Laibin Liu, Alan A. Alfieri, Dong Yu, Ekambar R. Kandimalla, Sudhir Agrawal, and Chandan Guha* PLoS One. 2012; 7(1): e29357. PMCID: PMC3251576; Gundersen H J, Bendtsen T F, Korbo L, Marcussen N, Moller A, Nielsen K, Nyengaard J R, Pakkenberg B, Sorensen F B, Vesterby A et al. (1988) Acta Pathol Microbiol Immunol Scand 96:379-394; Uma D P, Saini M R, Saharan B R, Bharatiya H C (1979) Radiat Res 80:214-220; Wu W, Onn A, Isobe T, Itasaka S, Langley R R, Shitani T, Shibuya K, Komaki R, Ryan A J, Fidler I J, Herbst R S, O'Reilly M S. Mol Cancer Ther. 2007 February; 6(2):471-83.; Mah V, Seligson D B, Li A, Márquez D C, Wistuba I I, Elshimali Y, Fishbein M C, Chia D, Pietras R J, Goodglick L. Cancer Res. 2007 Nov. 1; 67(21):10484-90.). Sections can then be stained for histologic examination of treatment effects; and used to do established assays for apoptosis (TUNEL), ERβ, ERα and EGFR expression (cf. Wu W, Onn A, Isobe T, Itasaka S, Langley R R, Shitani T, Shibuya K, Komaki R, Ryan A J, Fidler I J, Herbst R S, O'Reilly M S. Mol Cancer Ther. 2007 February; 6(2):471-83.; Mah V, Seligson D B, Li A, Marquez D C, Wistuba I I, Elshimali Y, Fishbein M C, Chia D, Pietras R J, Goodglick L. Cancer Res. 2007 Nov. 1; 67(21):10484-90.).

### 19. IN VIVO ANIMAL IMAGING TO ASSESS BIODISTRIBUTION OF A LABELED RADIATION MITIGATOR

A range of options have been identified for radiolabeling DPN (Harki D A, Satyamurthy N, Stout D B, Phelps M E, Dervan P B (2008). Proc Natl Acad Sci USA 2008 Sep. 2; 105(35):13039-44.; Shu C J, Campbell D O, Lee J T, Tran A Q, Wengrod J C, Witte O N, Phelps M E, Satyamurthy N, Czernin J, Radu C G (2010). J Nucl Med 51(7):1092-8. Stout D B, Chatziioannou A F, Lawson T P, Silverman R W, Gambhir S S, Phelps M E. Mol Imaging Biol. 2005 November-December; 7(6):393-402.).

Labeling with either ¹¹C or ¹⁴C may be used to obtain the important biodistribution data (Sepehr E, Lebl-Rinnova M, Mann M K, Pisani S L, Churchwell M I, Korol D L, Katzenellenbogen J A, Doerge D R (2012). J Pharm Biomed Anal. 71:119-26. Epub 2012 Aug. 24.).

Once DPN has been radiolabeled, the biodistribution of the labeled compound over 2-3 hours can be monitored in mice to determine where the activity targets and how it clears from tissues. Imaging may also be performed at additional time points up to 8 hours. The imaging center has 3 PET scanners for PET rodent research (Inveon DPET, Focus 220: Siemens Preclinical Solutions, and a Genisys4 PET/Xray, Sofie Biosciences) which may be used together with a rodent CT system (MicroCAT II, Siemens). After imaging is complete, the animals can be sacrificed and radioactivity measured in major organs using a gamma counter (Wizard3, Perkin-Elmer). From the in vivo biodistribution data, residency times may be determined and Olinda used to estimate radiation dosimetry in rodents. About 5-6 mice can be used for these experiments, with an additional group as needed to confirm findings using established methods (Harki D A, Satyamurthy N, Stout D B, Phelps M E, Dervan P B (2008). Proc Natl Acad Sci USA 2008 Sep. 2; 105(35):13039-44. Shu C J, Campbell D O, Lee J T, Tran A Q, Wengrod J C, Witte O N, Phelps M E, Satyamurthy N, Czernin J, Radu C G (2010). J Nucl Med 51(7):1092-8.; Stout D B, Chatziioannou A F, Lawson T P, Silverman R W, Gambhir S S, Phelps M E. Mol Imaging Biol. 2005 November-December; 7(6):393-402.; Fueger B J, Czernin J, Hildebrandt I, Tran C, Halpern B S, Stout D, Phelps M E, Weber W A (2006). J Nucl Med. 47(6):999-1006).

### 20. ADDITIONAL ERβ LIGAND STUDIES

An ERβ ligand such as DPN is an attractive radiation mitigator candidate because ERβ is expressed and active in many tissues that are well-known to be impacted by radiation injury (Krege J H, Hodgin J B, Couse J F, Enmark E, Warner M, Mahler J F, Sar M, Korach K S, Gustafsson J A, Smithies 0 (1998). Proc Natl Acad Sci USA 95:15677-15682; Harris H A (2007). Mol Endocrinol. 21(1):1-13. Epub 2006 Mar. 23. Review; Brush J, Lipnick S L, Phillips T, Sitko J, McDonald J T, McBride W H. Semin Radiat Oncol. 2007 April; 17(2):121-30). Since exposure to these ligands is limited to only a few days post-radiation, it is unlikely that undesirable effects of long-term exposure to estradiol-related ligands (which also activate ERα) will be encountered with this ERβ-selective agent (Rooks W H 2nd, Dorfman R1 (1961). Endocrinology. 68:838-43; Chlebowski R T, Anderson G L, Manson J E, Schwartz A G, Wakelee H, Gass M, Rodabough R J, Johnson K C, Wactawski-Wende J, Kotchen J M, Ockene J K, O'Sullivan M J, Hubbell F A, Chien J W, Chen C, Stefanick M L. J Natl Cancer Inst. 2010 Sep. 22; 102(18):1413-21. Epub 2010 Aug. 13). In general, ERβ appears to promote differentiated functions, anti-inflammatory and tissue-protective effects in response to various types of cell injury or irradiation (Krege J H, Hodgin J B, Couse J F, Enmark E, Warner M, Mahler J F, Sar M, Korach K S, Gustafsson J A, Smithies O (1998). Proc Natl Acad Sci USA 95:15677-15682; Harris H A (2007). Mol Endocrinol. 21(1):1-13. Epub 2006 Mar. 23. Review).

Studies can be performed to assess varying DPN doses and treatment schedules. An alternative radiation mitigator candidate for further development is ERB-041 (another high-affinity ERβ-binding ligand) which has notable oral bioavailability. (Brush J, Lipnick S L, Phillips T, Sitko J, McDonald J T, McBride W H. Semin Radiat Oncol. 2007 April; 17(2):121-30; Kim K, Damoiseaux R, Norris A J, Rivina L, Bradley K, Jung M E, Gatti R A, Schiestl R H, McBride W H. Int J Radiat Biol. 2011 August; 87(8):839-45. Epub 2011 Mar. 14. PubMed PMID: 21401317; PubMed Central PMCID: PMC3203687; Kim K, Pollard J M, Norris A J, McDonald J T, Sun Y, Micewicz E, Pettijohn K, Damoiseaux R, Iwamoto K S, Sayre J W, Price B D, Gatti R A, McBride W H. Clin Cancer Res. 2009 Dec. 1; 15(23):7238-45. PMCID: PMC2787903; Fu Q, Berbée M, Wang W, Boerma M, Wang J, Schmid H A, Hauer-Jensen M. Radiat Res. 2011 June; 175(6):728-35.).

Additional studies include ERβ knockout mice to complement ERβ knockdown studies and offer further proof of ERβ specificity of this action in vivo. As noted above, estradiol signaling depends on the balance between ERα and ERβ activity in a given tissue. ERβ^{-/-} mice have a phenotype different from that of ERα^{-/-} mice thus allowing better understanding of the role of ERβ. Generation of ERβ^{-/-} mice was described previously (Krege J H, Hodgin J B, Couse J F, Enmark E, Warner M, Mahler J F, Sar M, Korach K S, Gustafsson J A, Smithies O (1998). Proc Natl Acad Sci USA 95:15677-15682). As needed, heterozigous B6.129P2-Esr2^{tm1Unc}/J are available from the Jackson Laboratory. ERβ-null (ERβ^{-/-}) mice can be generated by breeding homozygous (ERβ^{-/-}) males with heterozygous (ERβ^{+/-}) females. Genotyping using PCR can be done on DNA isolated from tails of 2-week-old mice (Liu M, Oyarzabal E A, Yang R, Murphy S J, Hurn P D. J Neurosci Methods 2008 Jun. 30; 171(2):214-7. Epub 2008 Mar. 18). Experiments using the most effective agonist would be done, with tissues and blood obtained and analyzed as noted above.

ERβ ligand combined with G-CSF. Cytokines and growth factors, particularly those of the hematopoietic system, can also protect against radiation-induced injury, in part by increasing tissue cellularity and thus ensuring a larger number of surviving cells. Granulocyte-macrophage colony stimulating factor (GM-CSF) and granulocyte colony stimulating factor (G-CSF) are used to partially reconstitute the immune system in cancer patients after destruction of the bone marrow during cancer treatment, and may be effective both as radio-protectants and radiation-injury mitigators (76-79). Consensus groups have recommended therapies such as G-CSF for radiation-induced neutropenia (77). G-CSF (100 μg/kg SQ for 3-10 days) may also have additional benefits in responding to radiation damage in epithelial tissues (79). Hence, G-CSF treatment can be combined with an optimal ERβ ligand to assess combination effects on radiation mitigation after TBI.

In view of reports on some radioprotective activity by the weak ERβ-binder genistein, studies to investigate soy-free versus standard feed are ongoing (Landauer M R, Srinivasan V, Seed T M (2003). J Appl Toxicol. 23(6):379-85; Davis T A, Clarke T K, Mog S R, Landauer M R (2007). Int J Radiat Biol. 83(3):141-51; Raffoul J J, Wang Y, Kucuk O, Forman J D, Sarkar F H, Hillman G G (2006). BMC Cancer. 6:107).

### 21. PHARMACOKINETIC STUDIES OF ERβ LIGANDS

Plasma DPN concentrations in vivo can be determined using the LC-MS method described previously (67). Briefly, drug administration in mice can be followed by collection of blood samples (e.g., 100 μl) at the distal tail at each time point (0.15 min, 30 min and 1, 2, 4, 8 and 24 hrs). Thereafter, plasma samples can be prepared and diluted with citrate buffer pH 5.0 and subjected to supported liquid extraction on SLE plates (Biotage, Charlotte, N.C.). Methyl t-butylester extract is dried under nitrogen and redissolved in methanol/water (1:1; v/v) for LC-MS. DPN is then analyzed by reverse-phase HPLC coupled with mass spectrometry using negative-ion electrospray ionization (ESI) selected reaction monitoring (transitions, m/z 238 to 132, 238 to 211) on a triple-quadrupole mass analyzer (Agilent 6460). Conjugated DPN can be determined after treatment of plasma with glucuronidase/sulfatase as described (67). DPN concentrations can be interpolated from an internal standard curve constructed with signals obtained for deuterated DPN spiked into the experimental plasma samples (m/z 242 to 132 versus 238 to 132). Pharmacokinetic parameters can be determined using PK Solutions 2.0 software (SummitPK) yielding bioavailability parameters including time to peak concentration (tₘₐₓ), peak concentration (Cₘₐₓ), minimal concentration (Cₘᵢₙ), steady state concentration (Cₛₛ), volume of distribution (V_{d}), half life (t_{1/2}), total clearance (C₁) and area under the time-concentration curve (AUC). Assays for structural variants related to DPN can be constructed using appropriate ionization conditions and internal standards synthesized with deuterium. Bioavailability of DPN can be tested in mice either administered the compounds by subcutaneous, intravenous or oral routes.

### 22. STATISTICAL ANALYSIS

Analysis of variance (ANOVA) can be used to compare the outcome measures between experimental conditions. In cases of non-normality or non-constant variance we can investigate the use of data transformations (ex. log transform). For example, for the assays measuring ER transcripts and protein the ANOVA models can contain terms for radiation dose, day and the dose by day interaction effect. Data can be presented as means±SEM, except for post-irradiation duration of survival, which can be presented as the median survival with interquartile range as the measure of variability. ANOVA models can be used to compare quantitative outcomes between study groups. Survival curves can be constructed using the Kaplan-Meier method and can be compared using the log-rank test as appropriate (Kodell R L, Lensing S Y, Landes R D, Kumar K S, Hauer-Jensen M (2010). Biometrics 66:239-248. PMCID: PMC3036987). The Cox proportional hazards multiple regression method can be used to determine the influence of radiation mitigators such as DPN on post-irradiation survival across radiation doses. These models can contain terms for treatment, dose and the treatment by dose interaction effects. We plan to use 8 mice/group in preliminary studies to screen dosages and schedules. However, for the definitive studies with the final chosen agent, the sample size can be 24 mice in each group. Using an LD70/30 model, this is based on the assumption that controls can have 30% survival rate and treatment conditions can have 90% survival rate. Based on these assumptions a 0.050 level two-sided log-rank test for equality of survival curves can have 95% power to detect the difference between the groups. Differences can be considered statistically significant when the P value is less than 0.05.

### 23. EXAMPLE OVERVIEW

Use cell lines to identify ER-selective ligands might best promote recovery and survival of critical tissues post-radiation and the pathways involved, with a primary focus on DPN. ERβ-ligands DPN, ERB-041 and control ligands PPT, PHTPP are used for in vitro studies with human lung cells (e.g. H23, A549), HUVEC and colon cell lines (e.g. HCC-2998, and cells available from commercial suppliers) for ERβ expression levels and ligand interactions. Levels of ER transcripts & protein are assayed in vitro after irradiation and in cells treated with or without ERβ-ligands. ERβ may be up-regulated by irradiation as a protective tissue response. Tests for apoptosis are done by TUNEL assay, with confirmatory assay of 85-kD cleaved PARP protein by Western blot. Assays of changes in apoptosis mediators are conducted to explore mechanisms. Assays for clonogenicity and cell proliferation are performed. Modulation of post-RT cell cycle progression and DNA repair by ERβ ligands given 24 h post-RT are pursued to complete the picture of how ERβ modulates DNA repair. Increased growth factor production/secretion. ELISA methods are used to find effects of ERβ-ligands (24 h post-RT) on production of EGFR/VEGFR ligands in cells with/without irradiation. Decipher cell ER signaling modes after RT using ER siRNA's to suppress ERβ. Knock-down system is used to confirm that ERβ is an obligate pathway to mediate cell survival after RT. Develop ER-selective ligands (with a focus on DPN) as radiation mitigators administered after radiation exposure, with an emphasis on broad activity to reduce major morbidity and promote survival using animal models. Promising radiation mitigators detected in vitro are used for in vivo tests in mice exposed to TBI. ERβ1-agonist DPN are the primary focus of these studies. Perform survival experiments using animal models to assess ERβ-selective ligands as radiation mitigators; define the in vivo mitigator potency of DPN in terms of dose, routes of administration and scheduling. C3H and C57Bl/6 mice are irradiated using a Gamma cell 40 irradiator (Cs-137 source), then randomized to receive vehicle, DPN or ERB-041 at varying doses by s.c. or oral routes respectively, beginning 24 h or at later times after TBI and continued Qd for up to 5 days. Kaplan-Meier survival curves, median survival, lethality are recorded. In selected studies, mouse tissue samples are collected postmortem at the termination of experiments for further analyses. Agents that increase survival to >90% are tested further to establish their optimal dose and delivery schedule and for their dose modifying factor (DMF). Single doses are compared with multiple doses; s.c. and p.o. routes. High single doses are used to establish toxicity. To establish the DMF, mice are exposed to graded TBI doses over a range of doses from 7.725-9.5Gy in 0.5 Gy increments. Identify an agent with a DMF of at least 1.25 or higher (e.g. 1.5). Sampling of Systemic Blood and Mouse Tissues. Assays include estimates of blood cell counts, CFU assays, testing of isolated bone marrow cells, and selected tissue assays. Treatments provide evidence for increased recovery of hematopoietic cells and local tissue recovery.

### 24. REFERENCES

1. Pietras R J & Marquez-Garban D C. (2007) Clin Cancer Res. 13:4672-6.
2. Krege J H, et al. (1998) Proc Natl Acad Sci USA 95:15677-15682
3. Harris H A (2007). Mol Endocrinol. 21(1):1-13. Epub 2006 Mar. 23. Review.
4. Wada-Hiraike O, et al (2005) Br J Cancer 92:2286-91.
5. Wu X, et al (2001) J Biol Chem. 276:23962-8.
6. Becker K A, et al (2005) Oncogene 24(42):6345-53.
7. Torlakovic E, et al (2005) Int J Cancer 117(3):381-6.
8. Rooks W H 2nd, Dorfman R I (1961) Endocrinology 68:838-43.
9. Zhou Y & Mi M T (2005). J Radiat Res. 46(4):425-33.
10. Landauer M R, et al. (2003). J Appl Toxicol. 23(6):379-85.
11. Davis T A, et al. (2007). Int J Radiat Biol. 83(3):141-51.
12. Raffoul J J, et al. (2006). BMC Cancer 6:107.
13. Cristofaro P A, et al. (2006) Crit Care Med. August; 34(8):2188-93.
14. Brush J, et al. (2007) Semin Radiat Oncol. April; 17(2):121-30
15. Razandi M, et al. (1999) Mol Endocrinol. February; 13(2):307-19.
16. Acconcia F, et al. (2004) Biochem Biophys Res Commun. April 9; 316(3):878-83.
17. Pietras R J, et al. (2001) Endocrine. April; 14(3):417-27.
18. Song R X, et al. (2004) Proc Natl Acad Sci USA. February 17; 101(7):2076-81. Epub 2004 Feb. 5.
19. Stabile L P, et al. (2002) Cancer Res. April 1; 62(7):2141-50.
20. Pietras R J, et al. (2005) Steroids. May-June; 70(5-7):372-81. Epub 2005 Mar. 25.
21. Pietras R J, et al. (1999) Cancer Res. March 15; 59(6):1347-55.
22. Sturla L M, et al. (2005) J Biol Chem. April 15; 280(15):14597-604. Epub 2005 February
23. Das A K, et al. (2007) Cancer Res. June 1; 67(11):5267-74.
24. Marquez D C, et al. (2001) Endocrine. November; 16(2):73-81.
25. Pietras R J, et al. (2006) Breast Cancer Res Treatment 100, Suppl 1: 507.
26. Pietras R J, et al. (1995) Oncogene. June 15; 10(12):2435-46.
27. Li D, et al. (2002). Oncogene. 21(18):2805-14.
28. Petit A M, et al. (1997) Am J Pathol. December; 151(6):1523-30.
29. Pietras R J. (2003) Breast J. September-October;9(5):361-73.
30. Harrington W R, et al. (2003) Mol Cell Endocrinol. August 29; 206(1-2):13-22.
31. Márquez-Garbán D C, et al. (2007) Steroids. February; 72(2):135-43. Epub 2007 February 5.
32. Márquez D C, et al. (2006) Mol Cell Endocrinol. February 26; 246(1-2):91-100. Epub 2006 Jan. 4.
33. Kinyamu H & Archer T (2003) Mol Cell Biol. 23(16):5867-81.
34. Bond G L & Levine A J (2007) Oncogene February 26; 26(9):1317-23.
35. Mishra R G, et al. (2006) Am J Physiol Heart Circ Physiol. January; 290(1):H295-303. Epub 2005 Sep. 30.
36. Patisaul H B, et al. (2002) Endocrinology June; 143(6):2189-97.
37. Weihua Z, et al. (2001) Proc Natl Acad Sci USA. May 22; 98(11):6330-5.
38. Lau S K, et al. (2006) Appl Immunohistochem Mol Morphol. March; 14(1):83-7.
39. Kirsch E A, et al. (1999) Am J Respir Cell Mol Biol. April; 20(4):658-66.
40. Xu X, et al. (2012) Genome Med. April 30; 4(4):31.
41. Nübel T, et al. (2006) Clin Cancer Res. February 1; 12(3 Pt 1):933-9.
42. Sarkaria J N, et al. (1998) Cancer Res. October 1; 58(19):4375-82.
43. Aguilar Z, et al. (1999) Oncogene October 28; 18(44):6050-62
44. Kim K, et al. (2011) Int J Radiat Biol. August; 87(8):839-45. Epub 2011 Mar. 14.
45. Kim K, et al. (2009) Clin Cancer Res. December 1; 15(23):7238-45.
46. Maclachlan T, et al. (2005) Int J Radiat Biol. August; 81(8):567-79.
47. Kodell R L, et al. (2010 Biometrics 66:239-248.
48. Epperly M, et al. (2007) Radiat Res. November; 168(5):552-9.
49. Brown S L, et al. (2008) Radiat Res. April; 169(4):474-8).
50. Chen B J, et al. (2010) PLoS One 2010; 5:e11056.
51. Zhou D, et al. (2012) Int J Radiat Oncol Biol Bhys. 2010 Sep. 25, [Epub ahead of print].
52. Suman S, et al. (2012) Radiat Oncol. January 20; 7:6.
53. Subhrajit Saha, et al. (2012) PLoS One. 2012; 7(1): e29357.
54. Gundersen H J, et al. (1988) Acta Pathol Microbiol Immunol Scand 96:379-394
55. Uma D P, et al. (1979) Radiat Res 80:214-220
56. Wu W, et al. (2007) Mol Cancer Ther. February; 6(2):471-83.
57. Mah V, et al. (2007) Cancer Res. November 1; 67(21):10484-90.
58. Meyers M J, et al. (2001) J Med Chem. November 22; 44(24):4230-51.
59. Sawyer, J. S., et al. (1988) J. Am. Chem. Soc. 110, 842-53.
60. Harki D A, et al. (1988) Proc Natl Acad Sci USA September 2; 105(35):13039-44.
61. Shu C J, et al. (2010) J Nucl Med 51(7):1092-8.
62. Stout D B, et al. (2005) Mol Imaging Biol. November-December; 7(6):393-402.
63. Fueger B J, et al. (2006) J Nucl Med. 47(6):999-1006.
64. Chlebowski R T, et al. (2006) J Natl Cancer Inst. September 22; 102(18):1413-21.
65. Liu M, et al. (2008) J Neurosci Methods June 30; 171(2):214-7.
66. Fu Q, et al. (2011) Radit. Res. June; 175(6):728-35.
67. Sepehr E, et al. (2012) J Pharm Biomed Anal. 71:119-26. Epub 2012 Aug. 24.
68. Kim S, et al. (2013) Radiation Research. 180:259-267.
69. Wang B, et al. (2013) J Radiation Res. 54: 620-629.
70. Rios C I, ET AL. (2014) Drug Development Res. 75: 23-28.
71. Illing A, et al. (2012) Haematologica. 97:1131-5.
72. Shim G J, et al. (2003) Proc Natl Acad Sci USA. 100:6694-9.
73. Hamada H, et al. (2006) Circulation. 114:2261-70.
74. Dupont S, et al. (2000) Development. 127:4277-4291.
75. Saleiro D, et al. (2010) Cancer Prev Res (Phila). 3:1198-204.
76. U.S. Department of Health and Human Service. National Institutes of Health National Institute of Allergy and Infectious Diseases. NIH Strategic Plan and Research Agenda for Medical Countermeasures against Radiological and Nuclear Threats. June 2005; NIH Publication No. 05-560.
77. Weiss J F & Landauer M R. (2009) Int J Radiat Biol. 85:539-73.
78. Watanabe T, et al. (1996) Br J Haematol. 94:619-27.
79. Kim J S, et al. (2013) Arch Pharm Res. 36:1252-61.
80. McKendry L H. (1980) J Labelled Compounds Radiopharm 17: 43-51.
81. Christensen D M, (2014) Emerg Med Clin North Am. 32:245-65.
82. Dey P, et al. (2013) J Mol Endocrinol 51:T61-74.
83. Thomas C G, et al. (2011) Breast Cancer Res Treat 127:417-27.
84. Warner M & Gustafsson J A. (2010) Biochem Biophys Res Commun. 396:63-6.

TABLE 1
selected estrogen receptor β agonists

| | | | | | Highest | | | |
|---|---|---|---|---|---|---|---|---|
| | | | | | Phase | | | |
| Product | | Active | Therapeutic | | of | | Molecule | |
| Name | Other Names | Ingredient | Category | Condition Treated | Development | Product Type | Type | Company Name |
| AC186 | AC 186 | — | Immunology | Multiple Sclerosis | PreClinical | Investigational | — | Acadia |
| | | | and | (PC) | | Drug | | Pharmaceuticals |
| | | | Inflammation | | | | | Inc |
| AUS131 | AUS 131, S | — | Central Nervous | Benign Prostatic | Phase II | Investigational | — | Ausio |
| | equol | | System, | Hyperplasia(II), | | Drug | | Pharmaceuticals |
| | | | Dermatology, | Menopausal | | | | LLC |
| | | | Genitourinary | Disorders(II), | | | | |
| | | | Disorders, | Acne(II), Hormone | | | | |
| | | | Musculoskeletal, | Refractory Prostate | | | | |
| | | | Oncology | Cancer(I), | | | | |
| | | | | Neurodegenerative | | | | |
| | | | | Disorders(I), | | | | |
| | | | | Osteoporosis(I) | | | | |
| BAY865310 | BAY 865310, | — | Genitourinary | Menopausal | Phase II | Investigational | — | Bayer HealthCare |
| | BAY86 5310, | | Disorders | Disorders(II) | | Drug | | Pharmaceuticals, |
| | ZK 283197, | | | | | | | Bayer Ag |
| | ZK283197 | | | | | | | |
| ER Beta | — | — | Genitourinary | Menopausal | Research | Investigational | — | Bayer HealthCare |
| Agonist | | | Disorders | Disorders(R) | | Drug | | Pharmaceuticals |
| BAYER | | | | | | | | |
| SCHERING | | | | | | | | |
| ER-beta | — | — | Central Nervous | Neuropathic | PreClinical | Investigational | — | Acadia |
| agonist | | | System | Pain(PC), | | Drug | | Pharmaceuticals |
| ACADIA | | | | Parkinson's | | | | Inc |
| | | | | Disease(PC) | | | | |
| ERB041 | ERB 041 | prinaberel | Genitourinary | Endometriosis(II), | Phase II | Investigational | Small | Pfizer Inc |
| | | | Disorders, | Rheumatoid | | Drug | | |
| | | | Musculoskeletal | Arthritis(II) | | | | |
| ERB196 | ERB 196, | — | Immunology | Inflammatory | Discontinued | Investigational | — | Pfizer Inc |
| | WAY 202196, | | and | Disorders(D) | | Drug | | |
| | WAY202196 | | Inflammation | | | | | |
| Estrogen | ERbeta | — | Genitourinary | Endometriosis(PC), | PreClinical | Investigational | — | Radius Health Inc |
| Receptor | | | Disorders, | Inflammatory | | Drug | | |
| beta | | | Immunology | Disorders(PC) | | | | |
| agonist | | | and | | | | | |
| | | | Inflammation | | | | | |
| Eviendep | — | — | Endocrine, | Familial | Phase II | Investigational | — | Nestle Health |
| | | | Metabolic and | Adenomatous | | Drug | Science SA | |
| | | | Genetic | Polyposis(II) | | | | |
| | | | Disorders | | | | | |
| GTx878 | GTx 878 | — | Genitourinary | Benign Prostatic | PreClinical | Investigational | — | GTx Inc |
| | | | Disorders | Hyperplasia(PC), | | Drug | | |
| | | | | Chronic Pelvic Pain | | | | |
| | | | | Syndrome(PC) | | | | |
| KB9520 | ER beta, KB | — | Central Nervous | Benign Prostatic | PreClinical | Investigational | — | Karo Bio AB |
| | 9520, Selective | | System, | Hyperplasia(PC), | | Drug | | |
| | ER beta | | Genitourinary | Depression(PC), | | | | |
| | agonist | | Disorders, | Multiple | | | | |
| | | | Oncology | Sclerosis(PC), | | | | |
| | | | | Oncology(PC) | | | | |
| Menerba | MF 101, | liquiritigenin | Genitourinary | Menopausal | Discontinued | Investigational | Small | Bionovo Inc |
| | MF101 | | Disorders | Disorders(D) | | Drug | | |
| NDC1022 | NDC 1022 | — | Central Nervous | Multiple | PreClinical | Investigational | — | Endece, |
| | | | System | Sclerosis(PC) | | Drug | | ENDECE Neural |
| NDC1308 | NDC 1308 | — | Central Nervous | Multiple | PreClinical | Investigational | — | Endece, |
| | | | System, | Sclerosis(PC), | | Drug | | ENDECE Neural |
| | | | Oncology | Oncology(PC) | | | | |
| NDC1352 | NDC 1352 | — | Central Nervous | Inflammatory | PreClinical | Investigational | — | Endece |
| | | | System, | Disorders(PC), | | Drug | | |
| | | | Immunology | Pain(PC) | | | | |
| | | | and | | | | | |
| | | | Inflammation | | | | | |
| NDC1407 | NDC 1407 | — | Oncology | Oncology(PC) | PreClinical | Investigational | — | Endece |
| | | | | | | Drug | | |
| Neumune | HE 2100, | androstene | Central Nervous | Acute Radiation | Phase I | Investigational | Small | Harbor |
| | HE2100 | diol | System, | Injury(I), Multiple | | Drug | | Therapeutics Inc |
| | | | Infectious | Sclerosis(PC), | | | | |
| | | | Diseases, | Infectious | | | | |
| | | | Radiation Injury | Diseases(D) | | | | |
| Seala | VG 101, | — | Genitourinary | Postmenopausal | PreClinical | Investigational | — | Bionovo Inc |
| | VG101 | | Disorders | Atrophic | | Drug | | |
| | | | | Vaginitis(PC) | | | | |

TABLE 2
selected estrogen receptor β agonists

| | | | | | | | Mol- | |
|---|---|---|---|---|---|---|---|---|
| Product | Other | Active | Therapeutic | Condition | Current | | ecule | |
| Name | Names | Ingredient | Category | Treated | Phase | Product Type | Type | Companies |
| AC186 | AC 186 | — | Immunology | Multiple | PreClinical | Investigational | — | Acadia Pharmaceuticals |
| | | | and | Sclerosis | | Drug | | Inc\hat{ }(Originator, |
| | | | Inflammation | | | | | Developer) |
| AUS131 | AUS 131, S | — | Dermatology | Acne | Phase II | Investigational | — | Ausio Pharmaceuticals |
| | equol | | | | | Drug | | LLC\hat{ }(Primary Owner, |
| | | | | | | | | Developer)\hat{ }(Unknown |
| | | | | | | | | [II]) |
| AUS131 | AUS 131, S | — | Genitourinary | Benign | Phase II | Investigational | — | Ausio Pharmaceuticals |
| | equol | | Disorders | Prostatic | | Drug | | LLC\hat{ }(Primary Owner, |
| | | | | Hyperplasia | | | | Developer)\hat{ }(Australia [II], |
| | | | | | | | | India (Asia-Pacific) [II], |
| | | | | | | | | United States (North |
| | | | | | | | | America) [II]) |
| AUS131 | AUS 131, S | — | Oncology | Hormone | Phase I | Investigational | — | Ausio Pharmaceuticals |
| | equol | | | Refractory | | Drug | | LLC\hat{ }(Primary Owner, |
| | | | | Prostate Cancer | | | | Developer) |
| AUS131 | AUS 131, S | — | Genitourinary | Menopausal | Phase II | Investigational | — | Ausio Pharmaceuticals |
| | equol | | Disorders | Disorders | | Drug | | LLC\hat{ }(Primary Owner, |
| | | | | | | | | Developer)\hat{ }(Australia [II], |
| | | | | | | | | United States (North |
| | | | | | | | | America) [II]) |
| AUS131 | AUS 131, S | — | Central | Neuro- | Phase I | Investigational | — | Ausio Pharmaceuticals |
| | equol | | Nervous | degenerative | | Drug | | LLC\hat{ }(Primary Owner, |
| | | | System | Disorders | | | | Developer)\hat{ }(Unknown [I]) |
| AUS131 | AUS 131, S | — | Musculo- | Osteoporosis | Phase I | Investigational | — | Ausio Pharmaceuticals |
| | equol | | skeletal | | | Drug | | LLC\hat{ }(Primary Owner, |
| | | | | | | | | Developer)\hat{ }(Unknown [I]) |
| BAY865310 | BAY 865310, | — | Genitourinary | Menopausal | Phase II | Investigational | — | Bayer Ag\hat{ }(Primary |
| | BAY86 5310, | | Disorders | Disorders | | Drug | | Owner)\|Bayer HealthCare |
| | ZK 283197, | | | | | | | Pharmaceuticals\hat{ }(Originator, |
| | ZK283197 | | | | | | | Developer)\hat{ }(Germany |
| | | | | | | | | (Europe) [D], Netherlands |
| | | | | | | | | (Europe) [DI], United |
| | | | | | | | | Kingdom (Europe) [D]) |
| ER Beta | — | — | Genitourinary | Menopausal | Research | Investigational | — | Bayer HealthCare |
| Agonist | | | Disorders | Disorders | | Drug | | Pharmaceuticals\hat{ }(Primary |
| BAYER | | | | | | | | Owner, Developer) |
| SCHERING | | | | | | | | |
| ER-beta | — | — | Central | Neuropathic | PreClinical | Investigational | — | Acadia Pharmaceuticals |
| agonist | | | Nervous | Pain | | Drug | | Inc\hat{ }(Originator, |
| ACADIA | | | System | | | | | Developer) |
| ER-beta | — | — | Central | Parkinson's | PreClinical | Investigational | — | Acadia Pharmaceuticals |
| agonist | | | Nervous | Disease | | Drug | | Inc\hat{ }(Originator, |
| ACADIA | | | System | | | | | Developer) |
| ERB041 | ERB 041 | prinaberel | Genitourinary | Endometriosis | Phase II | Investigational | Small | Pfizer Inc\hat{ }(Primary |
| | | | Disorders | | | Drug | | Owner, |
| | | | | | | | | Developer)\hat{ }(United States |
| | | | | | | | | (North America) [D])\| |
| | | | | | | | | Wyeth\hat{ }(Primary Owner, |
| | | | | | | | | Developer) |
| ERB041 | ERB 041 | prinaberel | Musculo- | Rheumatoid | Phase II | Investigational | Small | Pfizer Inc\hat{ }(Primary |
| | | | skeletal | Arthritis | | Drug | | Owner, |
| | | | | | | | | Developer)\hat{ }(Canada |
| | | | | | | | | (North America) [D], |
| | | | | | | | | Europe [D], United States |
| | | | | | | | | (North America) [D])\| |
| | | | | | | | | Wyeth\hat{ }(Primary Owner, |
| | | | | | | | | Developer) |
| ERB196 | ERB 196, | — | Immunology | Inflammatory | Discontinued | Investigational | — | Pfizer Inc\hat{ }(Primary |
| | WAY | | and | Disorders | | Drug | | Owner, |
| | 202196, | | Inflammation | | | | | Developer)\|Wyeth\hat{ }(Primary |
| | WAY202196 | | | | | | | Owner, Developer) |
| Estrogen | ERbeta | — | Genitourinary | Endometriosis | PreClinical | Investigational | — | Radius Health |
| Receptor | | | Disorders | | | Drug | | Inc\hat{ }(Primary Owner, |
| beta | | | | | | | | Developer) |
| agonist | | | | | | | | |
| Estrogen | ERbeta | — | Immunology | Inflammatory | PreClinical | Investigational | — | Radius Health |
| Receptor | | | and | Disorders | | Drug | | Inc\hat{ }(Primary Owner, |
| beta | | | Inflammation | | | | | Developer) |
| agonist | | | | | | | | |
| Eviendep | — | — | Endocrine, | Familial | Phase II | Investigational | — | CM&amp; D Pharma |
| | | | Metabolic and | Adenomatous | | Drug | | Limited\hat{ }(Primary Owner, |
| | | | Genetic | Polyposis | | | | Developer)\hat{ }(Italy (Europe) |
| | | | Disorders | | | | | [II])\|Nestle Health Science |
| | | | | | | | | SA\hat{ }(Primary Owner, |
| | | | | | | | | Developer)\hat{ }(Italy (Europe) |
| | | | | | | | | [II]) |
| GTx878 | GTx 878 | — | Genitourinary | Benign | PreClinical | Investigational | — | GTx Inc\hat{ }(Originator, |
| | | | Disorders | Prostatic | | Drug | | Developer) |
| | | | | Hyperplasia | | | | |
| GTx878 | GTx 878 | — | Genitourinary | Chronic Pelvic | PreClinical | Investigational | — | GTx Inc\hat{ }(Originator, |
| | | | Disorders | Pain Syndrome | | Drug | | Developer) |
| KB9520 | ER beta, KB | — | Genitourinary | Benign | PreClinical | Investigational | — | Karo Bio AB\hat{ }(Originator, |
| | 9520, | | Disorders | Prostatic | | Drug | | Developer) |
| | Selective ER | | | Hyperplasia | | | | |
| | beta agonist | | | | | | | |
| KB9520 | ER beta, KB | — | Central | Depression | PreClinical | Investigational | — | Karo Bio AB\hat{ }(Originator, |
| | 9520, | | Nervous | | | Drug | | Developer) |
| | Selective ER | | System | | | | | |
| | beta agonist | | | | | | | |
| KB9520 | ER beta, KB | — | Central | Multiple | PreClinical | Investigational | — | Karo Bio AB\hat{ }(Originator, |
| | 9520, | | Nervous | Sclerosis | | Drug | | Developer) |
| | Selective ER | | System | | | | | |
| | beta agonist | | | | | | | |
| KB9520 | ER beta, KB | — | Oncology | Oncology | PreClinical | Investigational | — | Karo Bio AB\hat{ }(Originator, |
| | 9520, | | | | | Drug | | Developer) |
| | Selective ER | | | | | | | |
| | beta agonist | | | | | | | |
| Menerba | MF 101, | liquiritigenin | Genitourinary | Menopausal | Discontinued | Investigational | Small | Bionovo Inc\hat{ }(Originator, |
| | MF 101 | | Disorders | Disorders | | Drug | | Developer)\hat{ }(United States |
| | | | | | | | | [D]) |
| NDC1022 | NDC 1022 | — | Central | Multiple | PreClinical | Investigational | — | ENDECE Neural\hat{ }(Co- |
| | | | Nervous | Sclerosis | | Drug | | Developer)\| |
| | | | System | | | | | Endece\hat{ }(Originator, |
| | | | | | | | | Developer) |
| NDC1308 | NDC 1308 | — | Central | Multiple | PreClinical | Investigational | — | ENDECE Neural\hat{ }(Co- |
| | | | Nervous | Sclerosis | | Drug | | Developer)\| |
| | | | System | | | | | Endece\hat{ }(Originator, |
| | | | | | | | | Developer) |
| NDC1308 | NDC 1308 | — | Oncology | Oncology | PreClinical | Investigational | — | Endece\hat{ }(Originator, |
| | | | | | | Drug | | Developer) |
| NDC1352 | NDC 1352 | — | Immunology | Inflammatory | PreClinical | Investigational | — | Endece\hat{ }(Originator, |
| | | | and | Disorders | | Drug | | Developer) |
| | | | Inflammation | | | | | |
| NDC1352 | NDC 1352 | — | Central | Pain | PreClinical | Investigational | — | Endece\hat{ }(Originator, |
| | | | Nervous | | | Drug | | Developer) |
| | | | System | | | | | |
| NDC1407 | NDC 1407 | — | Oncology | Oncology | PreClinical | Investigational | — | Endece\hat{ }(Originator, |
| | | | | | | Drug | | Developer) |
| Neumune | HE 2100, | androstenediol | Radiation | Acute | Phase I | Investigational | Small | Harbor Therapeutics |
| | HE2100 | | Injury | Radiation | | Drug | | Inc\hat{ }(Primary Owner, |
| | | | | Injury | | | | Developer)\hat{ }(Netherlands |
| | | | | | | | | (Europe) [I], United States |
| | | | | | | | | (North America) [I]) |
| Neumune | HE 2100, | androstenediol | Infectious | Infectious | Discontinued | Investigational | Small | Harbor BioSciences, |
| | HE2100 | | Diseases | Diseases | | Drug | | Inc.\hat{ }(Originator, |
| | | | | | | | | Developer)\|Harbor |
| | | | | | | | | Therapeutics Inc\hat{ }(Primary |
| | | | | | | | | Owner, Developer) |
| Neumune | HE 2100, | androstenediol | Central | Multiple | PreClinical | Investigational | Small | Harbor Therapeutics |
| | HE2100 | | Nervous | Sclerosis | | Drug | | Inc\hat{ }(Primary Owner, |
| | | | System | | | | | Developer) |
| Seala | VG 101, | — | Genitourinary | Post | PreClinical | Investigational | — | Bionovo Inc\hat{ }(Originator, |
| | VG101 | | Disorders | menopausal | | Drug | | Developer) |
| | | | | Atrophic | | | | |
| | | | | Vaginitis | | | | |

TABLE 3
selected estrogen receptor β agonist active ingredients

| | | Chemical | | | Chemical/ | | | |
|---|---|---|---|---|---|---|---|---|
| Active | PubChem | Structure | | | Biological | | Molecular | Molecular |
| Ingredient | ID | Image | Products | Companies | Class | Chemical Name | Formula | Weight |
| androstenediol | 10634 | | 1 | 1 | Androstenes | (3S,8R,9S,10R,13S,14S,17S)- | C19H30O2 | 290.40 |
| | | | | | | 10,13-dimethyl- | | |
| | | | | | | 2,3,4,7,8,9,11,12,14,15,16,17- | | |
| | | | | | | dodecahydro-1H- | | |
| | | | | | | cyclopenta[a]phenanthrene-3,17- | | |
| | | | | | | diol | | |
| liquiritigenin | 114829 | | 1 | 1 | Benzopyrans, | (2S)-7-hydroxy-2-(4- | C15H12O4 | 256.30 |
| | | | | | Pyrans | hydroxyphenyl)-2,3- | | |
| | | | | | | dihydrochromen-4-one | | |
| prinaberel | 5326893 | | 1 | 1 | Azoles, Oxazoles | (4Z)-4-(7-ethenyl-5-hydroxy-3H- | C15H10FNO3 | 271.20 |
| | | | | | | 1,3-benzoxazol-2-ylidene)-2- | | |
| | | | | | | fluorocyclohexa-2,5-dien-1-one | | |

TABLE 4
selected estrogen receptor β agonist milestones

| Product | | | | | | | |
|---|---|---|---|---|---|---|---|
| Name | Company Name | Indication | Milestone Status | Milestone Class | Milestone Brief | Est End Date | Status |
| AUS131 | Ausio | Benign Prostatic | Revised | Trial Updates | Phase II trial result | Dec. 31, 2013 | Active |
| | Pharmaceuticals | Hyperplasia | | | | | |
| | LLC | | | | | | |
| AUS131 | Ausio | Benign Prostatic | Revised | Trial Updates | Phase IIa trial end | Dec. 31, 2013 | Active |
| | Pharmaceuticals | Hyperplasia | | | | | |
| | LLC | | | | | | |

TABLE 5
selected estrogen receptor β agonist references

| | | Highest | | | | | | | | |
|---|---|---|---|---|---|---|---|---|---|---|
| Product | Active | Phase of | Company | IPR | | Filing | Publication | Estimated | | |
| Name | Ingredient | Development | Name | Number | Pat. No. | Date | Date | Expiry Date | Patent Type | Country |
| AUS131 | — | II | CHILDREN'S | 7396855 | U.S. Pat. | Jul. 24, | Jul. 08, 2008 | Dec. 03, | Composition | United |
| | | | HOSPITAL | | No. | 2003 | | 2024 | | States |
| | | | MEDICAL | | 7,396,855 | | | | | |
| | | | CENTER; | | | | | | | |
| | | | AUSTRALIAN | | | | | | | |
| | | | HEALTH & | | | | | | | |
| | | | NUTRITION | | | | | | | |
| | | | ASSOC. LTD | | | | | | | |
| ERB041 | prinaberel | II | WYETH | 6794403 | U.S. Pat. | Dec. | Sep. 21, | Dec. 06, | Product | United |
| | | | | | No. | 04, 2002 | 2004 | 2022 | | States |
| | | | | | 6,794,403 | | | | | |
| Menerba | liquiritigenin | D | BIONOVO | 2008319051 | US | Jun. 22, | Dec. 25, | Oct. 09, | Composition | United |
| | | | INC | | 2008319051 | 2007 | 2008 | 2012 | | States |
| Menerba | liquiritigenin | D | BIONOVO | 7482029 | U.S. Pat. | Mar. 29, | Jan. 27, | Jan. 27, | Method of | United |
| | | | INC | | No. | 2006 | 2009 | 2013 | Use | States |
| | | | | | 7,482,029 | | | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating radiation damage in a patient in need of such treatment, said method comprising administering a therapeutically effective amount of an estrogen receptor β agonist to said patient.

2. The method of claim 1, wherein said estrogen receptor β agonist is selected from the group consisting of AC186, AUS131, BAY865310, 8β-VE2,8-vinylestra-1,3,5(10)-triene-3,17β-diol, AC74131, ERB041, ERB196, Eviendep, GTx878, KB9520, Menerba, NDC1022, NDC1308, NDC1352, NDC1407, Neumune, Seala, (3S,8R,9S,10R,13S,14S,17S)-10,13-dimethyl-2,3,4,7,8,9,11,12,14,15,16,17-dodecahydro-1H-cyclopenta[a]phenanthrene-3,17-diol, (2S)-7-hydroxy-2-(4-hydroxyphenyl)-2,3-dihydrochromen-4-one, (4Z)-4-(7-ethenyl-5-hydroxy-3H-1,3-benzoxazol-2-ylidene)-2-fluorocyclohexa-2,5-dien-1-one, and DPN (2,3-bis[4-hydroxyphenyl]-propionitrile).

3. The method of claim 1, wherein said radiation damage is associated with radiation therapy.

4. The method of claim 1, wherein said radiation damage is associated with exposure to nuclear material or radiological material.

5. The method of claim 1, wherein said radiation damage is to bone marrow, gastrointestinal tract, respiratory system, or cardiovascular system.

6. The method of claim 1, wherein said radiation damage is DNA damage.

7. The method of claim 1, wherein said estrogen receptor β agonist is

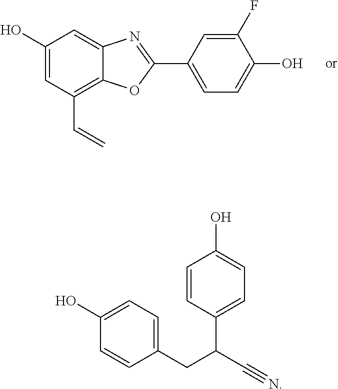 [SMILES: C=Cc1cc(O)cc2nc(-c3ccc(O)c(F)c3)oc12 ; N#CC(Cc1ccc(O)cc1)c1ccc(O)cc1]

8. The method of claim 1, wherein said estrogen receptor β agonist is 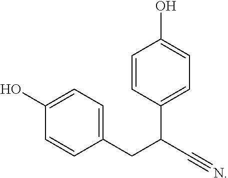 [SMILES: N#CC(Cc1ccc(O)cc1)c1ccc(O)cc1]
